(19) United States
(12) Patent Application Publication
(10) Pub. No.: US 2007/0078101 A1
(43) Pub. Date: Apr. 5, 2007

(54) Regulation of tgf-beta signaling by tomoregulin

(76) Inventors: Ali Hemmati Brivanlou, New York, NY (US); Chenbei Chang, Birmingham, AL (US)

Correspondence Address: KING & SPALDING, 1185 AVENUE OF THE AMERICAS, NEW YORK, NY, 10036-4003, US

(21) Appl. No.: 10/543,040

(22) PCT Filed: Jan. 21, 2004
(86) PCT No.: PCT/US04/01645

§ 371 Date: Aug. 17, 2006

Related U.S. Application Data
(60) Provisional application No. 60/441,625, filed on Jan. 21, 2003.

(51) Int. Cl. A61K 48/00; G01N 33/574; C12Q 1/68; C07K 14/82; A61K 39/395; C12N 5/08

(52) U.S. Cl. 514/44; 424/155.1; 514/12; 435/6; 435/7.23; 435/366

(57) ABSTRACT

The present invention provides methods of modulating TBF-β, particularly, nodal, Vg1 or BMP-2, but not activin, signalling by administration of a molecule that modulates the activity of TMEFF1. Diagnostic methods are also provided.

This application claims the benefit of United States Provisional Application No. 60/441,625, filed Jan. 21, 2003, which is incorporated herein by reference in its entirety.

This invention was made with Government support under grant number HD32105 awarded by the National Institutes of Health. The Government has certain rights in the invention.

### 1. TECHNICAL FIELD

The present invention relates to use of tomoregulin-1 (X7365) or TMEFF1 proteins and nucleic acids and molecules that modulate TMEFF to modulate TGF-β signalling. The invention further relates to therapeutic compositions and methods of diagnosis and therapy.

### 2. BACKGROUND OF THE INVENTION

TGF-β signaling has been implicated in multiple processes during early vertebrate development. Two main classes of TGFβ ligands play different roles in patterning of the early embryos (for reviews, see Hogan, 1996; Harland and Gerhart, 1997; Schier and Shen, 1999; Whitman, 2001). The activin/nodal/Vg1 subfamily participates in specification of endoderm and mesoderm in pre-gastrula embryos. At gastrula stages, ligands in this group are involved in dorsal mesoderm formation as well as anterior-posterior patterning. Later in development, these factors are part of the regulatory network to determine the left-right asymmetry of the vertebrate body axis and function to influence the dorsal-ventral patterning of the nervous system. Members of the second class of the TGFβ ligands, Bone Morphogenetic Proteins (BMPs), are involved mainly in ventralization of all germ layers in early embryos, resulting in suppression of neural and dorsal mesodermal cell fates. Subsequently, BMPs participate in formation and patterning of multiple tissues and organs, including the neural crest, heart, blood, kidney, limb, muscle and skeletal elements. The diverse activities of both groups of the TGFβ ligands are mediated by closely-related homologues in conserved signal transduction pathways, but they are subjected to differential regulation by other factors.

TGFβ signals are transduced inside the cells through two types of membrane serine-threonine kinase receptors. Upon binding to ligands, type II receptors phosphorylate type I receptors (activin receptor-like kinases, or ALKs), which then activate cytoplasmic signal transducers Smads. Activated Smads are translocated from cytoplasm to nucleus and cooperate with other transcription factors to influence gene expression (for reviews, see Derynck and Feng, 1997; Massague, 1998; Massague and Chen, 2000). Though the pathway is conserved for most TGFβ members, nodal signal transduction does require an additional component, the cripto/criptic/FRL1/one-eyed-pinhead protein (Schier and Shen, 1999; Shen and Schier, 2000; Whitman, 2001). The cripto family proteins contain a divergent EGF domain and a conserved CFC motif, and they function as co-receptors for nodal. In the absence of cripto, the type I receptor ALK4 can mediate signal transduction from activin, but not from nodal (Gritsman et al., 1999). Through direct binding to both ALK4 and nodal, cripto allows interaction of nodal with ALK4 to stimulate downstream responses (Reissman et al., 2001; Yeo and Whitman, 2001). Cripto is therefore a unique component in the TGFβ pathway, which may be required specifically for nodal-related ligands. Moreover, overexpression of Cripto has been implicated in breast cancer.

TGFβ signals can be regulated by factors at different cellular levels. For example, the naturally occurring truncated receptor BAMBI inhibits signaling by both classes of TGFβ ligands (Onichtchouk et al., 1999); and the inhibitory Smads, Smad6 and Smad7, block transduction of TGFβ signals in the cytoplasm (Nakao et al., 1997; Casellas and Hemmati-Brivanlou, 1998; Hata et al., 1998; Nakayama et al., 1998). The most prominent way of regulation of the TGFβ signals, however, is at the extracellular level. Many secreted factors are found to antagonize the activities of the TGFβ ligands in early embryos, and they include noggin, chordin, follistatin, Cerberus, Gremlin, Xnr3 and lefty (Smith and Harland, 1992; Hemmati-Brivanlou et al., 1994; Sasai et al., 1995; Piccolo et al, 1996; 1999; Zimmerman et al., 1996; Hansen et al., 1997; Hsu et al., 1998; Branford et al., 2000; Cheng et al., 2000; Tanegashima et al., 2000). Interestingly, though all these proteins can inhibit the function of BMPs, only a subset of them modulates activin/nodal activities. Cerberus blocks nodal but not activin signaling through direct binding to nodal (Piccolo et al., 1999); and lefty has been proposed to prevent interaction of activin/nodal with their receptors by occupying these receptors (Sakuma et al., 2002). Follistatin, the first identified secreted factor to regulate signals from the activin/nodal group of ligands, inhibits activin but)t not Vg1 through high affinity specific binding to activin (Kogawa et al., 1991; Fukui et al., 1993). These three regulators of activin/nodal signals do not share sequence homology among themselves, but several domain-specific homologous proteins to follistatin have been identified recently. There are three repetitive cystein-rich motifs, which are called follistatin (FS) modules, in follistatin. Proteins containing FS modules are identified from a variety of species. They contain either a single FS module, such as in follistatin-related proteins (FRPs, Mashimo et al., 1997; Okabayashi et al., 1999; De Groot et al., 2000) and follistatin-like (Flik, Patel et al., 1996; Towers et al., 1999), or they have two or more FS modules, such as in follistatin-related gene (FLRG, which has two FS modules. Hayette et al., 1998; or FSRP, Schneyer et al., 2001). Like follistatin, FLRG binds to activin with high affinity and blocks activin signaling (Schneyer et al., 2001; Tsuchida et al., 2001; Bartholin et al., 2002). These data suggest that proteins with FS modules may participate in regulation of activin/nodal activities.

Recently, a transmembrane protein with two FS modules is isolated from several species (Eib and Martens, 1996; Uchida et al., 1999; Eib et al., 2000; Da Silva et al., 2001). This protein, tomoregulin-1 (TMEFF1, previously named 7365), also contains an EGF motif in its extracellular region. The function of TMEFF1 in regulation of TGF-β signals has not been demonstrated. As demonstrated herein, unlike follistatin, TMEFF1 selectively inhibits nodal but not activin in Xenopus ectodermal explants. Both the FS and the EGF domains are necessary for the inhibitory function of TMEFF1. In addition, a soluble protein containing the FS modules and the EGF motif is not sufficient for nodal inhibition, and the membrane location of the protein is required.

TGFβ signalling is critical to a number of cellular and physiological processes and have been implicated in the induction and differentiation of mesodermal and endodermal tissues, for example, heart, muscle, kidney, liver, lung, pancreas, gut, etc., modulation of neural cell induction, growth and differentiation, modulation of epidermal cell induction, growth and/or differentiation, bone, cartilage and other connective tissue formation, regulation of cell proliferation, tumorigenesis, metastasis, adipogenesis, myogenesis, hematopoiesis, etc. Thus, proteins that modulate TGF-β activity have diagnostic and therapeutic uses. The present inventors have found that TMEFF1 regulates TGF-β signaling by inhibiting nodal but not activin activity. Thus, TMEFF1 may have a role in regulation of cellular proliferation, mesodermal and endodermal development, e.g., heart, lung, pancreas, liver, gut, etc. development and regulation.

Citation or identification of any reference in Section 2, or in any other section of this application, shall not be considered an admission that such reference is available as prior art to the present invention.

### 3. Summary of the Invention

The present invention is based upon the inventors' discovery that TMEFF1 inhibits nodal, Vg1, and BMP2 activities but not activin activity. TMEFF1 is shown to be expressed in a number of regions of the developing nervous system, including the diencephalon, midbrain, hindfrain, otic vesicle, cranial nerve placodes and dorsal trunk neural tissue. Overexpression of TMEFF1 in early Xenopus development results in reduction in or absence of head structures. Thus, the present invention relates to methods of regulating TGF-62 signalling, both in vitro and in vivo, using modulators of TMEFF1 activity, such as but not limited to, TMEFF1 proteins, and analogs, derivatives and fragments thereof, and TMEFF1 nucleic acids, including nucleic acids coding for TMEFF1 proteins, analogs, derivatives, and fragments, anti-TMEFF1 antibodies, anti-sense oligonucleotides, double stranded RNA for mediating RNAi, etc.

The invention in particular provides diagnostic and therapeutic methods using TMEFF1 proteins and nucleic acids, from any species, particularly, a mammal, such as a mouse, rat or, more particularly, human, as well as avian and amphibian TMEFF1. In particular embodiments, the TMEFF1 derived diagnostic or therapeutic agent used is of the same species as the animal to be treated or diagnosed.

The invention features methods using fragments of TMEFF1 nucleic acid molecules comprising or consisting of at least 480, 500, 550, 600, 650, 700, 750, 800, 850, 1000, 1100, 1200, 1300, 1400, 1500, 1600, 1700, 1800, 1900, 2000, 2100, 2200, 2300, 2400, 2500, 2600 or 2700 nucleotides, particularly fragments that encode one or, preferably, both FS domains and/or (preferably, and) the EGF domain of TMEFF1. In particular embodiments, the fragment encoded is soluble or, more preferably, membrane bound.

The invention features methods using isolated polypeptides or proteins which are encoded by a nucleic acid molecule having a nucleotide sequence that is at least about 50, 100, 150, 200, 250, 300, 400, 450, 500, 550, 600, 650 or more contiguous nucleotides of a TMEFF1 nucleotide sequence, wherein the polypeptides or proteins also exhibit at least one structural and/or functional feature of a polypeptide of the invention. In particular, the polypeptides or proteins contain one or, preferably, both, follistatin domains of TMEFF1 and, even more preferably, the EGF domain of TMEFF1. The protein may be soluble or membrane bound.

The methods of the invention involve, in specific embodiments, the polypeptides of the present invention, or biologically active portions thereof, operably linked to a heterologous amino-acid sequence to form fusion proteins, e.g. fusions to an F_{c} domain to increase in vivo half life of the protein or fusion to a heterologous transmembrane domain, secretion signal, etc.

The invention further provides diagnostic and therapeutic methods using anti-TMEFF1 antibodies, such as monoclonal or polyclonal antibodies or fragments thereof. Such anti-TMEFF1 antibodies can be conjugated antibodies comprising, for example, therapeutic or diagnostic agents. For example, the antibodies can be conjugated to a therapeutic moiety such as a chemotherapeutic cytotoxin, e.g., a cytostatic or cytocidal agent (e.g., paclitaxol, cytochalasin B or diphtheria toxin), an anti-angiogenic agent or a radioactive or fluorescent label.

In addition, in the methods of the invention, the TMEFF1 polypeptides or biologically active portions thereof, or anti-TMEFF1 antibodies or modulaters (inhibitors or activators) of TMEFF1, can be incorporated into pharmaceutical compositions, which optionally include pharmaceutically acceptable carriers.

In another aspect, the present invention provides methods of diagnosing a disease or disorder associated with aberrant TMEFF1 expression or activity and aberrant TGF-β (particularly, nodal, Vg1 or BMP-2) signaling which comprise the detection of the presence, activity or expression of TMEFF1 in a biological sample by contacting the biological sample with an agent capable of detecting an indicator of the presence (or, preferably the level) of TMEFF1 activity or expression such that the activity or expression of TMEFF1 is detected (or quantitated) in the biological sample. When compared to a control sample, such assays provide a diagnosis of a disease or disorder associated with aberrant TGF-β (particularly, nodal, Vg1 or BMP-2) signalling.

In another aspect, the invention provides methods for modulating TMEFF1 activity in order to modulate nodal, Vg1, BMP2, or other TGF-β signaling comprising contacting a cell with an agent that modulates (inhibits or stimulates) the activity or expression of TMEFF1 such that activity or expression in the cell is modulated. In one embodiment, the agent is an antibody that specifically binds to TMEFF1. In another embodiment, the agent is a TMEFF1 fragment, preferably comprising one or both FS domains and the EGF domains.

In another embodiment, the agent modulates expression of TMEFF1 by modulating transcription, splicing, or translation of an mRNA encoding TMEFF1. In yet another embodiment, the agent is a nucleic acid molecule having a nucleotide sequence that is antisense to the coding strand of an mRNA encoding TMEFF1 or is a double stranded RNA wherein one of the RNA strands is complementary to the coding strand of an mRNA encoding TMEFF1.

The present invention also provides methods to treat a subject having a disorder characterized by aberrant nodal, Vg1, BMP-2 or other TGF-β activity by administering an agent which is a modulator of TMEFF1 activity or a modulator of the expression of TMEFF1 to the subject. In one embodiment, the modulator is a TMEFF1 protein, or active fragment thereof. In another embodiment, the modulator is a TMEFF1 nucleic acid. In other embodiments, the modulator is an anti-TMEFF1 antibody, peptide, peptidomimetic, or other small molecule. In certain embodiments, the therapeutic and prophylactic methods of the invention involve the treatment or prevention of diseases or disorders in which the induction, growth or differentiation of mesodermal or endodermal cells or tissues (such as, but not limited to, heart, muscle, blood, liver, kidney, lungs, gut, pancreas, etc.) is desired to be promoted or inhibited; the induction, growth, or differentiation of neural, epidermal, bone, cartilege or other connective tissue is desired to be promoted or inhibited; or which involve cell hyperproliferation (e.g., cancer) or cell hypoproliferation. In a specific embodiment, TMEFF1 modulators are used to induce and/or direct the differentiation of stem cells, either adult or, preferably, embryonic stem cells (including human embryonic stem cells) into a mesodermal or endodermal cell type. Such differentiated cells can be used for therapeutic uses, e.g., tissue replacement therapy.

The present invention also provides diagnostic assays for identifying the presence or absence of a genetic lesion or mutation characterized by at least one of: (i) aberrant modification or mutation of a gene encoding TMEFF1, (ii) mis-regulation of a gene encoding TMEFF1, and (iii) aberrant post-translational modification of TMEFF1, wherein the diagnostic assay is for a disease or disorder associated with aberrant TGFβ signalling.

Other features and advantages of the invention will be apparent from the following detailed description and claims.

### 3.1. Definitions

“TMEFF1” is also called tomoregulin 1 or 7365, is a transmembrane protein containing two follistatin modules and a EGF domain in the extracellular region. The amino acid sequence of TMEFF1 has been determined for TMEFF1 in several species, see GenBank accession no. XP 237123 (human and rat), accession no. NP 057276 (human), accession no. CAB90827 (murine), accession no. BAA90820 (human), accession no. NP 003683 (human), accession no. CAA58792 (Xenopus laevis), all of which are incorporated by reference herein in their entireties. TMEFF1 also includes TMEFF1 orthologs from other species which are readily identified by methods well known to those skilled in the art, e.g., have a high degree of similarity to the human, rat and mouse TMEFF1 and two follistatin modules and an EGF domain. TMEFF1 proteins inhibit inhibition nodal, and, to a lesser extent, Vg1 and BMP-2 activity, but not activin activity.

The term “analog” as used herein refers to a polypeptide that possesses a similar or identical function as TMEFF1, e.g., having a TMEFF1 amino acid sequence, a fragment of TMEFF1, an anti-TMEFF1 antibody, or antibody fragment (or any other protein identified as a modulator of TMEFF1 ), but does not necessarily comprise a similar or identical amino acid sequence of TMEFF1, a fragment of TMEFF1, an anti-TMEFF1 antibody, or antibody fragment, or possess a similar~or identical structure of TMEFF1, a fragment of TMEFF1, an anti-TMEFF1 antibody, or antibody fragment. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of TMEFF1, a fragment of TMEFF1, an anti-TMEFF1 antibody, or antibody fragment described herein; (b) a polypeptide encoded by a nucleotide sequence that hybridizes under stringent conditions to a nucleotide sequence encoding TMEFF1, a fragment of TMEFF1, an anti-TMEFF1 antibody, or antibody fragment described herein of at least 5 amino acid residues, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding TMEFF1, an anti-TMEFF1 antibody, or antibody fragment described herein. A polypeptide with similar structure to TMEFF1, a fragment of TMEFF1, an anti-TMEFF1 antibody, or antibody fragment described herein refers to a polypeptide that has a similar secondary, tertiary or quaternary structure of TMEFF1, a fragment of TMEFF1, an anti-TMEFF1 antibody, or antibody fragment described herein. The structure of a polypeptide can determined by methods known to those skilled-in the art, including but not limited to, X-ray crystallography, nuclear magnetic resonance, and crystallographic electron microscopy.

The term “derivative” as used herein refers to a polypeptide that comprises an amino acid sequence of TMEFF1, a fragment of TMEFF1, an anti-TMEFF1 antibody, or antibody fragment (or any other protein identified as a modulator of TMEFF1) which has been altered by the introduction of amino acid residue substitutions, deletions or additions. The term “derivative” as used herein also refers to a TMEFF1 protein, a fragment of TMEFF1, an anti-TMEFF1 antibody, or antibody fragment (or any other protein identified as a modulator of TMEFF1) which has been modified, i.e, by the covalent attachment of any type of molecule to the polypeptide. For example, but not by way of limitation, TMEFF1, a fragment of TMEFF1, an anti-TMEFF1 antibody, or antibody fragment may be modified, e.g., by glycosylation, acetylation, pegylation, phosphorylation, amidation, derivatization by known protecting/blocking groups, proteolytic cleavage, linkage to a cellular ligand or other protein, etc. A derivative of TMEFF1, a fragment of TMEFF1, an anti-TMEFF1 antibody, or antibody fragment may be modified by chemical modifications using techniques known to those of skill in the art, including, but not limited to specific chemical cleavage, acetylation, formylation, metabolic synthesis of tunicamycin, etc. Further, a derivative of TMEFF1, a fragment of TMEFF1, an anti-TMEFF1 antibody, or antibody fragment may contain one or more non-classical amino acids. A polypeptide derivative possesses a similar or identical function as TMEFF1, a fragment of TMEFF1, an anti-TMEFF1 antibody, or antibody fragment described herein.

The term “epitopes” as used herein refers to portions of a TMEFF1 polypeptide (or any other protein identified as a modulator of TMEFF1) having antigenic or immunogenic activity in an animal, preferably a mammal, and most preferably in a human. An epitope having immunogenic activity is a portion of a TMEFF1 polypeptide that elicits an antibody response in an animal. An epitope having antigenic activity is a portion of a TMEFF1 polypeptide to which an antibody immunospecifically binds as determined by any method well known in the art, for example, by the immunoassays described herein. Antigenic epitopes need not necessarily be immunogenic.

The term “fragment” as used herein refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of TMEFF1 or an anti-TMEFF1 antibody (or any other protein identified as a modulator of TMEFF1).

An “isolated” or “purified” molecule (e.g., a protein, antibody, peptide, etc.) is substantially free of cellular material or other contaminating proteins from the cell or tissue source from which the protein is derived, or substantially free of chemical precursors or other chemicals when chemically synthesized. The language “substantially free of cellular material” includes preparations of a protein in which the protein is separated from cellular components of the cells from which it is isolated or recombinantly produced. Thus, an protein that is substantially free of cellular material includes preparations of proteins having less than about 30%, 20%, 10%, or 5% (by dry weight) of heterologous protein.(also referred to herein as a “contaminating protein”). When the protein is recombinantly produced, it is also preferably substantially free of culture medium, i.e., culture medium represents less than about 20%, 10%, or 5% of the volume of the protein preparation. When the protein or other molecule is produced by chemical synthesis, it is preferably substantially free of chemical precursors or other chemicals, i.e., it is separated from chemical precursors or other chemicals which are involved in the synthesis of the molecule. Accordingly such preparations of the protein or other molecule have less than about 30%, 20%, 10%, 5% (by dry weight) of chemical precursors or compounds other than the molecule of interest. In a preferred embodiment, antibodies, proteins and other molecules of the invention or fragments thereof are isolated or purified.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment, nucleic acid molecules encoding proteins of the invention are isolated or purified.

The term “antibodies or fragments that immunospecifically bind to TMEFF1” as used herein refers to antibodies or fragments thereof that specifically bind to a TMEFF1 polypeptide or a fragment of a TMEFF1 polypeptide and do not non-specifically bind to other polypeptides. Antibodies or fragments that immunospecifically bind to a TMEFF1 polypeptide or fragment thereof may have cross-reactivity with other antigens. Preferably, antibodies or fragments that immunospecifically bind to a TMEFF1 polypeptide or fragment thereof do not cross-react with other antigens. Antibodies or fragments that immunospecifically bind to an TMEFF1 polypeptide can be identified, for example, by immunoassays or other techniques known to those of skill in the art.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions x 100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, a “therapeutically effective amount” refers to that amount of the therapeutic agent sufficient to treat or manage a disease or disorder associated with aberrant TMEFF1 expression or activity and/or aberrant nodal and/or TGF-β activity. A therapeutically effective amount may refer to the amount of therapeutic agent sufficient to delay or minimize the onset of the disease or disorder. A therapeutically effective amount may also refer to the amount of the therapeutic agent that provides a therapeutic benefit in the treatment or management of the disease or disorder. Further, a therapeutically effective amount with respect to a therapeutic agent of the invention means that amount of therapeutic agent alone, or in combination with other therapies, that provides a therapeutic benefit in the treatment or management of such diseases or disorders.

As used herein, a “prophylactically effective amount” refers to that amount of the prophylactic agent sufficient to result in the prevention of a disease or disorder associated with aberrant TMEFF1 expression or activity and/or aberrant TGF-62 and/or nodal activity; including prevention of the recurrence or spread of such disease or disorder. A prophylactically effective amount with respect to a prophylactic agent of the invention means that amount of prophylactic agent alone, or in combination with other agents, that provides a prophylactic benefit in the prevention of such disease or disorder.

As used herein, the terms “therapeutic agent” and “therapeutic agents” refer to any agent(s) that can be used in the prevention, treatment, or management of a disease or disorder associated with aberrant TMEFF1 expression or activity and/or aberrant TGF-62 and/or nodal activity.

As used herein, the terms “therapies” and “therapy” can refer to any protocol(s), method(s) and or agent(s) that can be used in the prevention, treatment, or management of diseases or disorders associated with aberrant TMEFF1 expression and/or aberrant TGF-β and/or nodal activity.

As used herein, the terms “prophylactic agent” and “prophylactic agents” refer to any agent(s) that can be used in the prevention of the onset, recurrence or spread of a disease or disorder associated with aberrant TMEFF1 expression or activity and/or aberrant TGF-β and/or nodal activity.

As used herein, a “therapeutic protocol” refers to a regimen of timing and dosing of one or more therapeutic agents.

As used herein, a “prophylactic protocol” refers to a regimen of timing and dosing of one or more prophylactic agents.

A used herein, a “protocol” includes dosing schedules and dosing regimens.

As used herein, “in combination” refers to the use of more than one prophylactic and/or therapeutic agents.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

As used herein, the term “adjunctive” is used interchangeably with “in combination” or “combinatorial.” Such terms are also used where two or more therapeutic or prophylactic agents affect the treatment or prevention of the same disease.

As used herein, the terms “manage”, “managing” and “management” refer to the beneficial effects that a subject derives from a prophylactic or therapeutic agent, which does not result in a cure of the disease. In certain embodiments, a subject is administered one or more prophylactic or therapeutic agents to “manage” a disease so as to prevent the progression or worsening of the disease.

As used herein, the terms “prevent”, “preventing” and “prevention” refer to the prevention of the recurrence, spread or onset of a disease in a subject resulting from the administration of a prophylactic or therapeutic agent.

As used herein, underscoring or italicizing the name of a gene shall indicate the gene, in contrast to its encoded protein product, which is indicated by the name of the gene in the absence of any underscoring or italicizing. For example, “TMEFF1 ” shall mean the TMEFF1 gene, whereas “TMEFF1” shall indicate the protein product of the TMEFF1 gene

As used herein, the terms “treat”, “treating” and “treatment” refer to the eradication, reduction or amerlioration or symptons of a disease or disorder.

### 4. BRIEF DESCRIPTION OF THE FIGURES

FIGS. 1A and B. Differential regulation of TGF-β ligands by follistatin-module-containing molecules. A) TMEFF1 inhibits nodal but not activin activity in Xenopus ectodermal explants. Vg1 and BMP2 are also inhibited with less efficiency by TMEFF1. B) Follistatin (XFS), FLRG and TMEFF1 display differential inhibitory spectrums over TGFβ ligands. While both XFS and FLRG inhibit activin, TMEFF1 does not affect mesoderm induction by activin. TMEFF1, however, blocks nodal and Vg1 function. The doses of RNAs used are: TMEFF1, 2 ng; follistatin (XFS), 2 ng; human FLRG, 2 ng; activin, 5 pg; AXnr1 (Xenopus nodal), 500 pg; AVg1, 500 pg; BMP2, 500 pg. The RNAs were injected into the animal region of two cell stage embryos, and the animal caps were dissected at blastula stages (stage 9). The caps were collected at gastrula stages (stage 11, panel A) or tailbud stages (stage 28, panel B) for RT-PCR assays of gene expression patterns.

FIGS. 2A and B. Membrane location of TMEFF1 is required for its nodal inhibitory function. A) Schematic representation of the deletion mutants used in this study. SS, signal sequence; FS, follistatin modules; EGF, EGF domain; TM, transmembrane region. Though no protein was detected in conditioned medium from oocytes that were injected with wild type TMEFF1 RNA, injection of either TMEFF1-FS or TMEFF1-ΔTC RNA led to secretion of the mutant proteins from Xenopus ooccytes (not shown). B) Membrane location of TMEFF1 is important for nodal inhibition. Neither the mutant protein containing the FS modules alone nor the secreted protein containing the entire extracellular domain of TMEFF1 is sufficient to block nodal. Deletion of the cytoplasmic tail, however, does not abolish the inhibitory activity of TMEFF1. 2 ng RNA was injected for TMEFF1 and all the mutants, and 500 pg of AXnr1 was used.

FIGS. 3A and B. Both the FS modules and the EGF motif are required for nodal inhibition. A) Schematic representation of the mutant proteins used. B) Deletion of either the follistatin modules or the EGF domain impairs the ability of TMEFF1 to block nodal. The doses of RNAs used m this experiment: AXnr1, 500 pg; TMEFF1 and all the deletion mutants, 2 ng.

FIGS. 4A and B. The FS modules in TMEFF1 are not critical to determine the ligand specificity over nodal. A) Schematic representation of the chimeric protein constructed. In XFS-TMEFF1, the FS modules of TMEFF1 are replaced with the FS modules of XFS. B) The chimeric protein XFS-TMEFF1 inhibits both activin and nodal activities. The doses of TNAs used are: activin, 5 pg; AXnr1, 500 pg; TMEFF1, 2 ng; XFS, 2 ng; XFS-TMEFF1, 2 ng.

FIG. 5. Ectopic expression of TMEFF1 interferes with anterior development of early Xenopus embryos. 2 ng TMEFF1 RNA was injected into two dorsal or two ventral blastomeres of four-cell stage embryos; and the injected embryos were analyzed at tailbud stages for morphological changes. While ventral expression of TMEFF1 leads to mild tail defect, dorsal expression of TMEFF1 results in reduction of anterior structures - a phenotype similar to that induced by overexpression of a dominant negative nodal ligand.

FIGS. 6A and B. Temporal and spatial expression of TMEFF1 during early Xenopus development. A) TMEFF1 is expressed from mid gastrula stages onward. B) TMEFF1 is expressed in the neural plate at early neurula stages. As neurulation proceeds, its expression is enriched in the neural fold and the dorsal neural tube. At tailbud stages, TMEFF1 is detected in the diencephalons, midbrain, hindbrain, otic vesicles, cranial nerve plascodes and the trunk dorsal neural tissue.

### 5. DETAILED DESCRIPTION OF THE INVENTION

The present inventors have discovered that the protein TMEFF1 inhibits the activity of certain TGF-β's, such as nodal, BMP-2 and Vg1 but does not affect the activity of other TGF-β's, such as activin. Thus, TMEFF1 plays an important physiological role in cell proliferation, mesodermal and endodermal differentiation, e.g., differentiation of such tissues as lung, liver, kidney, pancreas, gut, stomach, blood, muscles, and any other mesodermal or endodermal derivatives, and may also play a role in nervous tissue formation and differentiation, epidermal growth and differentiation, growth and differentiation of bone and cartilage and other connective tissues.

The present invention provides therapeutic and diagnostic methods and compositions based on TMEFF1 proteins and nucleic acids, and analogs, derivatives and fragments thereof, and on anti-TMEFF1 antibodies. The invention provides for treatment of disorders of by administering compounds that promote TMEFF1 activity (e.g. TMEFF1 proteins and functionally active analogs and derivatives (including fragments) thereof; nucleic acids encoding the TMEFF1 proteins, analogs, or derivatives, agonists of TMEFF1).

The invention also provides methods of treatment of such diseases and disorders, for example, cancers and other hyperproliferative disorders, by administering compounds that antagonize, or inhibit, TMEFF1 function (e.g., antibodies, TMEFF1 antisense nucleic acids, TMEFF1 ribozymes).

Animal models, diagnostic methods and screening methods for predisposition to disorders are also provided by the invention.

For clarity of disclosure, and not by way of limitation, the detailed description of the invention is divided into the subsections that follow.

5.1 Obtaining TMEFF1 Proteins and Nucleic Acid

Since the nucleotide sequence of TMEFF1 from humans, rats, mice and Xenopus are known (see GenBank accession no. XP 237123 (human and rat), accession no. NP 057276 (human), accession no. CAB90827 (murine), accession no. BAA90820 (human), accession no. NP 003683 (human), accession no. CAA58792 (Xenopus laevis), all of which are incorporated by reference herein in their entireties), routine methods, such as the polymerase chain reaction, hybridization to a cDNA library from a source known to contain TMEFF1, chemical synthesis, etc, may be used to obtain nucleic acids encoding TMEFF1. Any eukaryotic cell can potentially serve as the nucleic acid source for the molecular cloning of the TMEFF1 gene. The nucleic acid sequences encoding TMEFF1 can be isolated from vertebrate, mammalian, human, porcine, bovine, feline, avian, equine, canine, murine, amphibia, preferably Xenopus , fish, e.g., zebrafish, fugu, as well as additional primate sources, insects, plants, etc. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, for example, Sambrook et al., 2001, Molecular Cloning, A Laboratory Manual, Third Edition, Cold Spring Harbor Laboratory Press, N.Y.; Ausubel et al., 1989, Current Protocols in Molecular Biology, Green Publishing Associates and Wiley Interscience, N.Y.; and Glover, D. M. (ed.), 1985, DNA Cloning: A Practical Approach, MRL Press, Ltd., Oxford, U.K. Vol. I, II, each of which is hereby incorporated by reference in its entirety). Clones derived from genomic DNA may contain regulatory and intron DNA regions in addition to coding regions; clones derived from cDNA will contain only exon sequences. Whatever the source, the gene should be molecularly cloned into a suitable vector for propagation of the gene.

The nucleotide sequence coding for a TMEFF1 protein or a functionally active analog or fragment or other derivative thereof, can be inserted into an appropriate expression vector, i.e., a vector that contains the necessary elements for the transcription and translation of the inserted protein-coding sequence. The necessary transcriptional and translational signals can also be supplied by the native TMEFF1 gene and/or its flanking regions or preferably, a heterologous promoter. A variety of host-vector systems may be utilized to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used

Any of the methods known in the art for the insertion of DNA fragments into a vector may be used to construct expression vectors containing a chimeric gene consisting of appropriate transcriptional/translational control signals and the protein coding sequences. These methods may include in vitro recombinant DNA and synthetic techniques and in vivo recombinants (genetic recombination). Expression of nucleic acid sequence encoding a TMEFF1 protein or peptide fragment may be regulated by a second nucleic acid sequence so that the TMEFF1 protein or peptide is expressed in a host transformed with the recombinant DNA molecule. For example, expression of a TMEFF1 protein may be controlled by any promoter/enhancer element known in the art. In a specific embodiment, the promoter is not a native TMEFF1 gene promoter. Promoters that may be used to control TMEFF1 expression include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); see also “Useful proteins from recombinant bacteria” in Scientific American, 1980, 242:74-94; plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region, which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region, which is active in pancreatic beta cells (Hanahan, 1985, Nature 315:115-122), immunoglobulin gene control region, which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region, which is active in testicular, breast, lymphoid and mast cells (Leder et al., 1986, Cell 45:485-495), albumin gene control region, which is active in liver (Pinkert et al., 1987, Genes and Devel. 1:268-276), alpha-fetoprotein gene control region, which is active in liver (Krumlauf et al., 1985, Mol. Cell. Biol. 5:1639-1648; Hammer et al., 1987, Science 235:53-58; alpha 1-antitrypsin gene control region, which is active in the liver (Kelsey et al., 1987, Genes and Devel. 1:161-171), beta-globin gene control region, which is active in myeloid cells (Mogram et al., 1985, Nature 315:338-340; Kollias et al., 1986, Cell 46:89-94; myelin basic protein gene control region, which is active in oligodendrocyte cells in the brain (Readhead et al., 1987, Cell 48:703-712); myosin light chain-2 gene control region, which is active in skeletal muscle (Sani, 1985, Nature 314:283-286), and gonadotropic releasing hormone gene control region, which is active in the hypothalamus (Mason et al., 1986, Science 234:1372-1378).

In a specific embodiment, a vector is used that comprises a promoter operably linked to a TMEFF1 -encoding nucleic acid, one or more origins of replication, and, optionally, one or more selectable markers (e.g., an antibiotic resistance gene).

In a specific embodiment, an expression construct is made by subcloning a TMEFF1 coding sequence into the EcoRI restriction site of each of the three pGEX vectors (Glutathione S-Transferase expression vectors; Smith and Johnson, 1988, Gene 7:31-40). This allows for the expression of the TMEFF1 protein product from the subclone in the correct reading frame.

Expression vectors containing TMEFF1 gene inserts can be identified by three general approaches: (a) nucleic acid hybridization, (b) presence or absence of “marker” gene functions, and (c) expression of inserted sequences. In the first approach, the presence of a TMEFF1 gene inserted in an expression vector can be detected by nucleic acid hybridization using probes comprising sequences that are homologous to an inserted TEMFF1 gene. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a TMEFF1 gene in the vector. For example, if the TMEFF1 gene is inserted within the marker gene sequence of the vector, recombinants containing the TMEFF1 insert can be identified by the absence of the marker gene function. In the third approach, recombinant expression vectors can be identified by assaying the TMEFF1 product expressed by the recombinant. Such assays can be based, for example, on the physical or functional properties of the TMEFF1 protein in in vitro assay systems, e.g., modulation of neural induction, modulation of epidermal tissue induction, modulation of bone, cartilage or other connective tissue induction, binding with anti-TMEFF1 antibody, etc.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors that can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen that modulates the expression of the inserted sequences, or modifies and processes the gene product in the specific fashion desired. Expression from certain promoters can be elevated in the presence of certain inducers; thus, expression of the genetically engineered TMEFF1 protein may be controlled. Furthermore, different host cells have characteristic and specific mechanisms for the translational and post-translational processing and modification (e.g., glycosylation, phosphorylation of proteins. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may effect processing reactions to different extents.

In other specific embodiments, the TMEFF1 protein, fragment, analog, or derivative may be expressed as a fusion, or chimeric protein product (comprising the protein, fragment, analog, or derivative joined via a peptide bond to a heterologous protein sequence (of a different protein)). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g. by use of a peptide synthesizer.

Both cDNA and genomic sequences can be cloned and expressed.

5.2. TMEFF1 Gene Products

In particular aspects, the methods of the invention use TMEFF1 protein, preferably human TMEFF1, and fragments and derivatives thereof that are functionally active (e.g., inhibit nodal, Vg1, and/or BMP-2 signaling), as well as nucleic acid sequences encoding the foregoing.

In specific embodiments, the invention utilizes fragments of a TMEFF1 protein consisting of at least 6 amino acids, 10 amino acids, 50 amino acids, or of at least 75 amino acids. In other embodiments, the proteins comprise or consist essentially of a TMEFF1 FS domain or two TMEFF1 FS domains and/or a TMEFF1 EGF domain or any combination of the foregoing, of a TMEFF1 protein. Fragments, or proteins comprising fragments, lacking some or all of the foregoing regions of a TMEFF1 protein can also be used as can nucleic acids encoding the foregoing.

The TMEFF1 protein may be isolated and purified by standard methods including chromatography (e.g., ion exchange, affinity, and sizing column chromatography), centrifugation, differential solubility, or by any other standard technique for the purification of proteins. The functional properties may be evaluated using any suitable assay (see Section 5.5). Alternatively, the protein can be synthesized by standard chemical methods known in the art (e.g., see Hunkapiller, M., et al., 1984, Nature 310:105-111).

In another alternate embodiment, native TMEFF1 proteins can be purified from natural sources, by standard methods such as those described above (e.g., immunoaffinity purification).

5.3. Generation of Antibodies to TMEFF1 Proteins and Derivatives Thereof

According to the invention, TMEFF1 protein, its fragments or other derivatives, or analogs thereof, may be used as an immunogen to generate antibodies that immunospecifically bind such an immunogen. In a specific embodiment, antibodies to a human TMEFF1 protein are used in methods of the invention. In specific embodiments, antibodies to the extracellular domain, one or more of the FS domains and/or the EGF domain of a TMEFF1 protein are produced. In a specific embodiment, fragments of a TMEFF1 protein identified as hydrophilic are used as immunogens for antibody production.

Various procedures known in the art may be used for the production of polyclonal antibodies to a TMEFF1 protein or derivative or analog. In a particular embodiment, rabbit polyclonal antibodies to an epitope of a TMEFF1 protein can be obtained. For the production of antibody, various host animals can be immunized by injection with the native TMEFF1 protein, or a synthetic version, or derivative (e.g., fragment) thereof, including but not limited to rabbits, mice, rats, etc. Various adjuvants maybe used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

Antibodies of the invention include, but are not limited to, monoclonal antibodies, synthetic antibodies, recombinantly produced antibodies, multispecific antibodies, human antibodies, humanized antibodies, chimeric antibodies, single-chain Fvs (scFv), single chain antibodies, Fab fragments, F(ab') fragments, disulfide-linked Fvs (sdFv), and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to TMEFF1. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies used in the methods of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies used in the methods of the present invention may be monospecific, bispecific, trispecific or of greater multispecificity. Multispecific antibodies may immunospecifically bind to different epitopes of a TMEFF1 polypeptide or may immunospecifically bind to both a TMEFF1 polypeptide as well a heterologous epitope, such as a heterologous polypeptide or solid support material. See, e.g., International Publication Nos. WO 93/17715, WO 92/08802, WO 91/00360, and WO 92/05793; Tutt, et al., 1991, J Immunol. 147:60-69; U.S. Pat. Nos. 4,474,893, 4,714,681, 4,925,648, 5,573,920, and 5,601,819; and Kostelny et al., 1992, J Immunol. 148:1547-1553.

The antibodies used in the methods of the invention include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides antibodies of the invention or fragments thereof that comprise a framework region known to those of skill in the art. Preferably, the antibody of the invention or fragment thereof is human or humanized.

In certain embodiments, the antibody to be used with the invention binds to an intracellular epitope, i.e., is an intrabody. An intrabody comprises at least a portion of an antibody that is capable of immunospecifically binding an antigen and preferably does not contain sequences coding for its secretion. Such antibodies will bind antigen intracellularly. In one embodiment, the intrabody comprises a single-chain Fv (“sFv”). sFvs are antibody fragments comprising the V_{H} and V_{L} domains of antibody, wherein these domains are present in a single polypeptide chain. Generally, the sFv polypeptide further comprises a polypeptide linker between the V_{H} and V_{L} domains which enables the sFv to form the desired structure for antigen binding. For a review of sFvs see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, WA, 1998, “Intrabodies: Basic Research and Clinical Gene Therapy Applications” Springer:New York). Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J Mol. Biol. 291:1119-1128; Ohage et al., 1999, J: Mo. Biol. 291:1129-1134; and Wirtz and Steipe, 1999, Protein Science 8:2245-2250, which references are incorporated herein by reference in their entireties. Recombinant molecular biological techniques such as those described for recombinant production of antibodies infra) may also be used in the generation of intrabodies.

Monoclonal antibodies can be prepared using a wide variety of techniques known in the art including the use of hybridoma, recombinant, and phage display technologies, or a combination thereof For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with TMEFF1 (either the full length protein or a domain or other fragment thereof) and once an immune response is detected, e.g., antibodies specific for TMEFF1 are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Hybridoma clones are then assayed by methods known in the art for cells that secrete antibodies capable of binding a polypeptide of the invention. Ascites

Human, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with TMEFF1 or fragment thereof with myeloma cells and then screening the hybridomas resulting from the fusion for hybridoma clones that secrete an antibody able to bind TMEFF1.

Antibody fragments which recognize specific TMEFF1 epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to the EphA2 epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J Immunol. Methods 182:41-50; Ames et al., 1995, J Immunol Methods 184:177; Kettleborough et al., 1994, Eur. J Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

Phage may be screened for TMEFF1 binding, or other TMEFF1-related activity, such as modulation of TMEFF1-mediated inhibition of TGF-β, BMP or wnt signalling.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12:864; Sawai et al., 1995, AJRI 34:26; and Better et al., 1988, Science 240:1041 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, PNAS 91:969), and chain shuffling (U.S. Pat. No. 5,565,332). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann et al., 1988, Nature 332:323, which are incorporated herein by reference in their entireties.)

5.3.1. Polynucleotides Encoding an Antibody

Anti-TMEFF1 antibodies may be recombinantly expressed using polynucleotides that encode the antibody. The polynucleotides may be obtained, and the nucleotide sequence of the polynucleotides determined, by any method known in the art (e.g., by cloning or amplifying the nucleic acids encoding the heavy to light chain antibody, e.g., from the hybridoma, and sequencing the nucleic acids encoding the heavy and light chains). Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J Mol. Biol. 278: 457-479 for a listing of human framework regions). Preferably, the polynucleotide generated by the combination of the framework regions and CDRs encodes an antibody that specifically binds to EphA2. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

5.3.2. Recombinant Expression of an Antibody

Recombinant expression of an anti-TMEFF1 antibody, or derivative, analog or fragment thereof (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, BioTechnology 8:2).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, PNAS 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthine guanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt- cells, respectively. Also, antimetabolite resistance can be used as the basis of selection for the following genes: dhfr, which confers resistance to methotrexate (Wigler et al., 1980, PNAS 77:357; O'Hare et al., 1981, PNAS 78:1527); gpt, which confers resistance to mycophenolic acid (Mulligan & Berg, 1981, PNAS 78:2072); neo, which confers resistance to the aminoglycoside G-418 (Wu and Wu, 1991, Biotherapy 3:87; Tolstoshev, 1993, Ann. Rev. Pharmacol. Toxicol. 32:573; Mulligan, 1993, Science 260:926; and Morgan and Anderson, 1993, Ann. Rev. Biochem. 62: 191; May, 1993, TIB TECH 11:155-); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre- Garapin et al., 1981, J Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, PNAS 77:2197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

The present invention encompasses methods using anti-TMEFF1 antibodies or fragments thereof recombinantly fused or chemically conjugated (including both covalent and non-covalent conjugations) to a heterologous polypeptide (or portion thereof, preferably to a polypeptide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., PCT publication WO 93/21232; EP 439,095; Naramura et al., Immunol. Lett. 39:91-99 (1994); U.S. Pat. No. 5,474,981; Gillies et al., PNAS 89:1428-1432 (1992); and Fell et al., J. Immunol. 146:2446-2452(1991), which are incorporated by reference in their entireties.

The present invention further includes compositions comprising heterologous polypeptides fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; PCT publication Nos. WO 96/04388 and WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341(1992) (said references incorporated by reference in their entireties).

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, anti-TMEFF1 antibodies or fragments or variants thereof conjugated to a diagnostic or detectable agent. Such antibodies can be useful for detecting, monitoring or prognosing the development or progression of a disease or disorder associated with aberrant TMEFF1 expression. Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I,), carbon (¹⁴C), sulfur (³⁵S), tritium (³H), indium (¹¹⁵In, ¹¹³In,¹¹²¹¹¹In,), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, and nonradioactive paramagnetic metal ions.

An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, gluticocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an antibody or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNFα, TNFβ, AIM I (see, International Publication No. WO 97/33899), AIM II (see, International Publication No. WO 97/34911), Fas Ligand (Takahashi et al., 1994, J. Immunol., 6:1567-1574), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, an antibody can be conjugated to therapeutic moieties such as a radioactive metal ion, such as alph-emiters such as ²¹³Bi or macrocyclic chelators useful for conjugating radiometal ions, including but not limited to, ¹³¹In, ^{13I}LU, ¹³¹Y, ¹³¹Ho, ¹³¹Sm, to polypeptides. In certain embodiments, the macrocyclic chelator is 1,4,7,10-tetraazacyclododecane-N,N′,N″,N″′ -tetraacetic acid (DOTA) which can be attached to the antibody via a linker molecule. Such linker molecules are commonly known in the art and described in Denardo et al., Clin Cancer Res. 4(10):2483-90 (1998); Peterson et al. Bioconjug. Chem. 10(4):553-7 (1999); and Zimmerman et al., Nucl. Med. Biol. 26(8):943-50 (1999) each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

5.4. TMEFF1 Protein Derivatives and Analogs

The invention further provides therapeutic and diagnostic methods using TMEFF1 proteins, and derivatives (including, but not limited to, fragments) and analogs of TMEFF1 proteins. In a specific embodiment, the derivative or analog is functionally active, i.e., capable of exhibiting one or more functional activities associated with a full-length, wild-type TMEFF1 protein. As one example, such derivatives or analogs that have the desired immunogenicity or antigenicity can be used, for example, in immunoassays, for immunization, for inhibition of TMEFF1 activity, etc. Derivatives or analogs that retain, or alternatively lack or inhibit, a desired TEFF1 property of interest can be used as inducers, or inhibitors, respectively, of such property and its physiological correlates. A specific embodiment provides a TMEFF1 fragment that can be bound specifically by an anti-TMEFF1 antibody. Derivatives or analogs of TMEFF1 can be tested for the desired activity by procedures known in the art, including but not limited to the assays described in Section 5.5.

In particular, TMEFF1 derivatives can be made by altering TMEFF1 sequences by substitutions, additions or deletions that provide for functionally equivalent molecules. These TMEFF1 include, but are not limited to, those containing, as a primary amino acid sequence, all or part of the amino acid sequence of a TMEFF1 protein including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

In a specific embodiment of the invention, proteins consisting of or comprising a fragment of a TMEFF1 protein consisting of at least 10 (continuous) amino acids of the TMEFF1 protein is provided. In other embodiments, the fragment consists of at least 20 or 50 amino acids of the TMEFF1 protein. In specific embodiments, such fragments are not larger than 35, 100 or 200 amino acids. Derivatives or analogs of TMEFF1 include but are not limited to those molecules comprising regions that are substantially homologous to TMEFF1 or fragments thereof (e.g., in various embodiments, at least 60% or 70% or 80% or 90% or 95% identity over an amino acid sequence of identical size or when compared to an aligned sequence in which the alignment is done by a computer homology program known in the art) or whose encoding nucleic acid is capable of hybridizing to a coding TMEFF1 sequence, under stringent, moderately stringent, or nonstringent conditions.

The TMEFF1 derivatives and analogs can be produced by various methods known in the art. The manipulations that result in their production can occur at the gene or protein level. For example, the cloned TMEFF1 gene sequence can be modified by any of numerous strategies known in the art (Sambrook et al., 2001, Molecular Cloning, A Laboratory Manual, Third Edition, Cold Spring Harbor Laboratory Press, N.Y.; Ausubel et al, 1989, Current Protocols in Molecular Biology, Green Publishing Associates and Wiley Interscience, N.Y.). The sequence can be cleaved at appropriate sites with restriction endonuclease(s), followed by further enzymatic modification if desired, isolated, and ligated in vitro. In the production of the gene encoding a derivative or analog of TMEFF1, care should be taken to ensure that the modified gene remains within the same translational reading frame as TMEFF1, uninterrupted by translational stop signals, in the gene region where the desired TMEFF1 activity is encoded.

Additionally, the TMEFF1-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, chemical mutagenesis, in vitro site-directed mutagenesis (Hutchinson, C., et al., 1978, J. Biol. Chem 253:6551), use of TAB® linkers (Pharmacia), etc.

Manipulations of the TMEFF1 sequence may also be made at the protein level. Included within the scope of the invention are TMEFF1 protein fragments or other derivatives or analogs that are differentially modified during or after translation, e.g. by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

In addition, analogs and derivatives of TMEFF1 can be chemically synthesized. For example, a peptide corresponding to a portion of a TMEFF1 protein that comprises the desired domain or that mediates the desired activity in vitro, can be synthesized by use of a peptide synthesizer. Furthermore, if desired, nonclassical amino acids or chemical amino acid analogs can be introduced as a substitution or addition into the TMEFF1 sequence. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, , β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

In a specific embodiment, the TMEFF1 derivative is a chimeric, or fusion, protein comprising a TMEFF1 protein or fragment thereof (preferably consisting of at least a domain or motif of the TMEFF1 protein, or at least 10 amino acids of the TMEFF1 protein) joined at its amino- or carboxy-terminus via a peptide bond to an amino acid sequence of a different protein. In one embodiment, such a chimeric protein is produced by recombinant expression of a nucleic acid encoding the protein (comprising a TMEFF1-coding sequence joined in-frame to a coding sequence for a different protein). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. Chimeric genes comprising portions of TMEFF1 fused to any heterologous protein-encoding sequences may be constructed. A specific embodiment provides a chimeric protein comprising a fragment of TMEFF1 of at least six amino acids.

One useful fusion protein is a GST fusion protein in which the polypeptide of the invention is fused to the C-terminus of GST sequences. Such fusion proteins can facilitate the purification of a recombinant polypeptide of the invention.

In another embodiment, the fusion protein contains a heterologous signal sequence at its N-terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family particularly all or part of a constant domain (or F_{c} fragment of an immunoglubin, e.g., an IgG (see e.g., U.S. Pat. No. 5,116,964 by Cupon et al.)). The immunoglobulin fusion proteins of the invention can be incorporated into pharmaceutical compositions and administered to a subject. The immunoglobulin fusion protein can be used to affect the bioavailability of a polypeptide of the invention. Moreover, the immunoglobulin fusion proteins of the invention can be used as immunogens to produce antibodies directed against a polypeptide of the invention in a subject.

In another specific embodiment, the TMEFF1 derivative is a molecule comprising a region of homology with a TMEFF1 protein. By way of example, in various embodiments, a first protein region can be considered “homologous” to a second protein region when the amino acid sequence of the first region is at least 30%, 40%, 50%, 60%, 70%, 75%, 80%, 90%, or 95% identical, when compared to any sequence in the second region of an equal number of amino acids as the number contained in the first region or when compared to an aligned sequence of the second region that has been aligned by a computer homology program known in the art. For example, a molecule can comprise one or more regions homologous to a TMEFF1 FS or EGF domain or to the extracellular domain of TMEFF1.

The TMEFF1 polypeptides of the invention can also be conjugate to a heterologous moiety, e.g., as described for antibodies in 5.3, supra.

5.5. Assays of TMEFF1 Proteins, Derivatives and Analogs

The functional activity of TMEFF1 proteins, derivatives and analogs can be assayed by various methods.

For example, in one embodiment, where one is assaying for the ability to bind or compete with wild-type TMEFF1 for binding to anti-TMEFF1 antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In another embodiment, where a TMEFF1-binding protein is identified, the binding can be assayed, e.g., by means well-known in the art. In another embodiment, physiological correlates of TMEFF1 binding to its substrates (e.g., modulation of signal transduction) can be assayed.

In addition a molecule can be tested for inhibition or promotion of TMEFF1 activity by assaying for nodal, Vg1 or BMP-2 activity, e.g. as disclosed in Section 6. In particular embodiments, the molecule may be tested for lack of modulation of activin signaling, e.g., as also disclosed in Section 6. For example, the molecule (or the mRNA encoding it) may be injected with TMEFF1 (or further co-injected with nodal, Vg1 or BMP-2) into Xenopus embryos at the two or four cell stage, and the animal caps explanted and assayed for an alteration in TMEFF1 activity, particularly nodal, Vg1 or BMP-2 signalling. Alternatively, the molecule may be assayed for the ability to rescue or increase the effects of overexpression of TMEFF1 (which, as described in Section 6 below, truncates anterior structures) in early Xenopus embryos.

Other methods will be known to the skilled artisan and are within the scope of the invention.

5.6. Therapeutic Uses

The invention provides for treatment or prevention of various diseases and disorders by administration of a therapeutic compound (termed herein “Therapeutic”). Such “Therapeutics” include but are not limited to: TMEFF1 proteins and functionally active analogs and derivatives (including fragments) thereof (e.g., as described hereinabove); antibodies thereto (as described hereinabove); peptidomimetics; nucleic acids encoding the TMEFF1 proteins, analogs, or derivatives (e.g., as described hereinabove); TMEFF1 antisense nucleic acids, and ribozymes and TMEFF1 agonists and antagonists, e.g., small molecules. Disorders involving mesodermal or endodermal tissue growth and/or differentiation (such as, but not limited to, gut, blood, lung, pancreas, kidney, muscle, liver tissue), cell proliferation (e.g., in cancer and other hyperproliferative or hypoproliferative disorders) and also potentially in neural tissue growth and/or differentiation, epidermal tissue growth and/or differentiation, bone or cartilage or other connective tissue growth and/or differentiation, or other disease or disorder associated with abnormal nodal, Vg1, BMP-2 or other TGF-β modulated by TMEFF1 are treated or prevented by administration of a Therapeutic that modulates TMEFF1 function. Disorders in which inhibition of mesodermal or endodermal tissue induction is desired or inhibition of epidermal induction and/or growth is desired, or promotion of cell proliferation are desired are treated or prevented by administration of a Therapeutic that induces, increases or upregulates TMEFF1 function. Diseases and disorders in which mesodermal or endodermal tissue growth or differentiation is deficient and/or desired, epidermal growth is desired, and inhibition of neural, bone, or cartilage tissue is desired, or inhibition of cell proliferation or transformed cell phenotype is desired are treated or prevented by administration of a Therapeutic that reduces or inhibits TMEFF1 Function. The above is described in detail in the subsections below.

Generally, administration of products of a species origin or species reactivity (in the case of antibodies) that is the same species as that of the patient is preferred. Thus, in a preferred embodiment, a human TMEFF1 protein, derivative, or analog, or nucleic acid, or an antibody to a human TMEFF1 protein, is therapeutically or prophylactically administered to a human patient.

In another embodiment, administration of a Therapeutic of the invention may be used to inhibit a BMP-2, nodal or Vg1 signaling pathway.

In another embodiment, a Therapeutic of the invention may be administered to cultured cells, preferably primary cells, to induce differentiation of the cells in culture into a desired tissue, e.g., a mesodermal or endodermal tissue, such as but not limited to, blood, lung, kidney, liver, pancreas, gut, muscle, and those differentiated cells (or resulting tissue) can be introduced into a patient as a therapeutic. In a preferred embodiment, adult or embryonic stem cells (preferably embryonic mammalian stem cells, more preferably, mouse or human embryonic stem cells) are contacted with and/or cultured in the presence of a Therapeutic of the invention (preferably, a TMEFF1 inhibitor) to induce differentiation of the stem cells into mesodermal or endodermal cells and, in some embodiments, ultimately, a specific mesodermal or endodermal tissue. Such cells or tissue can then be used for therapeutic purposes. In an even more preferred embodiment, the embryonic (or adult) stem cells are cultured in the absence of (and, preferably, are from a line of cells that have never been exposed to) feeder cells or cell extracts or other potential sources of contamination by infectious agents, e.g., have been cultured and maintained in the presence of a GSK-3 inhibitor such as, but not limited to, 6-bromoindirubin 3'oxime. In particular embodiments, the cells are engineered to produce a protein of interest, for example, a protein with therapeutic efficacy.

5.6.1. Cell Proliferation Disorders

Cancers and related disorders that can be treated or prevented by methods and compositions of the present invention include but are not limited to the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Conplete Book of Cancer Diagnosis, Treatment, and Recovery, Viking Penguin, Penguin Books U.S.A., Inc., United States of America). In a preferred embodiment, the methods of the invention are used in the treatment of breast cancer.

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal orignin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the ovary, bladder, breast, colon, lung, skin, pancreas, or uterus. In other specific embodiments, sarcoma, melanoma, or leukemia is treated or prevented.

In some embodiments, therapy by administration of one or more monoclonal antibodies is combined with the administration of one or more therapies such as, but not limited to, chemotherapies, radiation therapies, hormonal therapies, and/or biological therapies/immunotherapies.

In a specific embodiment, the methods of the invention encompass the administration of one or more angiogenesis inhibitors such as but not limited to: Angiostatin (plasminogen fragment); antiangiogenic antithrombin m; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; NMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD612; ZD 6474; farnesyl transferase inhibitors (FTI); and bisphosphonates.

Additional examples of anti-cancer agents that can be used in the various embodiments of the invention, including pharmaceutical compositions and dosage forms and kits of the invention, include, but are not limited to: acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decarbazine, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin 2 (including recombinant interleukin 2, or rIL2), interferon alpha-2a, interferon alpha-2b, interferon alpha-n1, interferon alpha-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nitrosoureas, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride. Other anti-cancer drugs include, but are not limited to: 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogens, antiestrogens, antineoplaston, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin m derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspernine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, dihydrotaxol, dioxamycin, diphenyl spiromustine, docetaxel, docosanol, dolasetron, doxifluridine, droloxifene, dronabinol, duocarmycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, fiezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors,. gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapnistone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein. kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, taxol, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thioguanine, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, and zinostatin stimalamer. Preferred additional anti-cancer drugs are 5-fluorouracil and leucovorin.

The invention also encompasses administration of the EphA2 antibodies of the invention in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physician's Desk Reference (56^{th} ed., 2002).

5.6.2. Treatment and Prevention of Disorders Involving Modulation or Endodermal or Mesodermal Tissue Induction, Growth or Maintenance.

The Therapeutics of the invention may be used in the treatment and prevention of diseases and disorder in which the growth, differentiation or maintenance of mesodermal or endodermal tissue is desired. In particular embodiments, the growth, differentiation or maintenance of heart, blood, muscle, blood, liver, kidney, pancreas, gut, or lung tissue is desired. In other specific embodiments, the inhibition or reduction in heart, blood, muscle, blood, liver, kidney, pancreas, gut, or lung tissue is desired. Such diseases and disorders may include, heart diseases and disorders, anemia, muscular degeneration diseases, cirrhosis, Hepatitis C infection and other liver disorders, kidney failure, diabetes, inflammatory bowel disease, COPD, etc. Molecules that promote TMEFF1 activity may be administered to inhibit or reduce mesodermal or endodermal tissue growth, differentiation, or maintenance. Molecules that inhibit TMEFF1 activity may be administered to promote mesodermal or endodermal tissue growth, differentiation or maintenance.

5.6.3. Treatment and Prevention of Disorders Involving Neural, Epidermal or Bone Cell Growth, Differentiation or Maintenance

Diseases and disorders in which the neural, epidermal, bone, cartilege or other connective tissue growth, differentiation or maintenance are desired to be promoted, in certain embodiments, and inhibited in other embodiments, may be treated by administration of Therapeutics of the invention. In particular, molecules that promote TMEFF1 function (and, thereby, inhibit nodal, Vg1 and BMP-2 signalling) may be useful in inducing neural cell growth, differentiation or maintenance and inhibiting epidermal or bone cell growth, differentiation or maintenance. On the other hand, molecules that inhibit TNMFFI function (and, thereby, promote nodal, Vg1 and BMP-2 signalling) may be useful in inhibiting neural cell growth, differentiation or maintenance and promoting epidermal or bone cell growth differentiation or maintenance.

In specific embodiments, the invention provides methods of treating (including ameliorating the symptoms of) injury to nervous tissue or a disease or disorder associated with damage to, degeneration of or defects in nervous tissue in a patient. In specific embodiments, the methods of the invention are used to treat injuries to spinal cord, brain, or peripheral nervous tissue, for example, but not limited to, tramatic brain injury, ischemic injury, spinal cord injury, etc.

In other embodiments, the disease or disorder that is treated is a neurodegenerative disease, such as but not limited to amyotrophic lateral sclerosis, Parkinson's disease, Huntington's chorea, multiple system atrophy, progressive supranuclear palsy, etc. The neurodegenerative disease may be associated with a bacterial, viral or other infection, such as damage caused by HIV or herpes viral infections, encephalitis, and Creutzfeldt-Jacob disease and kuru or may be due to the effects of a drug or toxin.

Methods of the invention also include methods of promoting wound healing and reducing scar formation, treating or preventing psoriasis, and other skin disorders, etc. In particular embodiments, the invention provides for the treatment or prevention of: (i) traumatic lesions, including lesions caused by physical injury or associated with surgery; (ii) ischemic lesions, in which a lack of oxygen results in cell injury or death, e.g., myocardial or cerebral infarction or ischemia, or spinal cord infarction or ischemia; (iii) malignant lesions, in which cells are destroyed or injured by malignant tissue; (iv) infectious lesions, in which tissue is destroyed or injured as a result of infection, for example, by an abscess or associated with infection by human immunodeficiency virus, herpes zoster, or herpes simplex virus or with Lyme disease, tuberculosis, syphilis; (v) degenerative lesions, in which tissue is destroyed or injured as a result of a degenerative process, including but not limited to nervous system degeneration associated with Parkinson's disease, Alzheimer's disease, Huntington's chorea, or amyotrophic lateral sclerosis; (vi) lesions associated with nutritional diseases or disorders, in which tissue is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration; (vii) lesions associated with systemic diseases including but not limited to diabetes or systemic lupus erythematosus; (viii) lesions caused by toxic substances including alcohol, lead, or other toxins; and (ix) demyelinated lesions of the nervous system, in which a portion of the nervous system is destroyed or injured by a demyelinating disease including but not limited to multiple sclerosis, human immunodeficiency virus-associated myelopathy, transverse myelopathy or various etiologies, progressive multifocal leukoencephalopathy, and central pontine myelinolysis.

5.6.4. Gene Therapy

In a specific embodiment, nucleic acids comprising a sequence encoding a TMEFF1 protein or functional derivative thereof, are administered to promote or inhibit TMEFF I function, by way of gene therapy. Gene therapy refers to therapy performed by the administration of a nucleic acid to a subject. In this embodiment of the invention, the nucleic acid produces its encoded protein that mediates a therapeutic effect by promoting or inhibiting TMEFF1 function.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology that can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, the Therapeutic comprises a TMEFF1 nucleic acid that is part of an expression vector that expresses a TMEFF1 protein or fragment or chimeric protein thereof in a suitable host. In particular, such a nucleic acid has a promoter operably linked to the TMEFF1 coding region, said promoter being inducible or constitutive, and, optionally, tissue-specific. In another particular embodiment, a nucleic acid molecule is used in which the TMEFF1 coding sequences and any other desired sequences are flanked by regions that promote homologous recombination at a desired site in the genome, thus providing for intrachromosomal expression of the TMEFF1 nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide that is known to enter the nucleus, by administering it in linkage to a ligand subject to receptor-mediated endocytosis (see e.g., Wu and Wu, 1987, J. Biol. Chem. 262:4429-4432) (that can be used to target cell types specifically expressing the receptors), etc.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains the TMEFF1 nucleic acid is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The TMEFF1 nucleic acid to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234.

Adeno-associated virus (AAV) has also been proposed for use in gene therapy (Walsh et al., 1993, Proc. Soc. Exp. Biol. Med; 204:289-300.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., cord blood cells,hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; embryonic stem cells, various stem or progenitor cells, in particular, hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a TMEFF1 nucleic acid is introduced into the cells such that it is expressible by the cells or their progeny, and the recombinant cells are then administered in vivo for therapeutic effect. In a specific embodiment, stem or progenitor cells are used. Any stem and/or progenitor cells that can be isolated and maintained in vitro can potentially be used in accordance with this embodiment of the present invention. Such stem cells include, but are not limited to, embryonic stem cells, hematopoietic stem cells (HSC), stem cells of epithelial tissues such as the skin and the lining of the gut, embryonic heart muscle cells, liver stem cells (PCT Publication WO 94/08598, dated Apr. 28, 1994), and neural stem cells (Stemple and Anderson, 1992, Cell 71:973-985).

Epithelial stem cells (ESCs) or keratinocytes can be obtained from tissues such as the skin and the lining of the gut by known procedures (Rheinwald, 1980, Meth. Cell Bio. 21A:229). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Rheinwald, 1980, Meth. Cell Bio. 21A:229; Pittelkow and Scott, 1986, Mayo Clinic Proc. 61:771). If the ESCs are provided by a donor, a method for suppression of host versus graft reactivity (e.g., irradiation, drug or antibody administration to promote moderate immunosuppression) can also be used.

With respect to hematopoietic stem cells (HSC), any technique that provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see, e.g., Kodo et al., 1984, J. Clin. Invest. 73:1377-1384). In a preferred embodiment of the present invention, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. USA 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Additional methods that can be adapted for use to deliver a nucleic acid encoding a TMEFF1 protein or functional derivative thereof are described in Section 5.8.

5.6.5. Targeted Reduction of TMEFF1 Gene Expression

5.6.5.1. Antisense Regulation of TMEFF1 Expression

In a specific embodiment, TMEFF1 function is inhibited by use of TMEFF1 antisense nucleic acids. The present invention provides the therapeutic or prophylactic use of nucleic acids of at least six nucleotides that are antisense to a gene or cDNA encoding TMEFF1 or a portion thereof. A TMEFF1 “antisense” nucleic acid as used herein refers to a nucleic acid capable of hybridizing to a portion of a TMEFF RNA (preferably mRNA) by virtue of some sequence complementarity. The antisense nucleic acid may be complementary to a coding and/or noncoding region of a TMEFF1 MnRNA. Such antisense nucleic acids have utility as Therapeutics that inhibits TMEFF1 function, and can be used in the treatment or prevention of disorders as described supra in Section 5.6.6.2 and its subsections.

The antisense nucleic acids of the invention can be oligonucleotides that are double-stranded or single-stranded, RNA or DNA or a modification or derivative thereof, which can be directly administered to a cell or can be produced intracellularly by transcription of exogenous, introduced sequences.

In a specific embodiment, the TMEFF1 antisense nucleic acids provided by the instant invention can be used to promote regeneration or growth (larger size).

The invention further provides pharmaceutical compositions comprising an effective amount of the TMEFF1 antisense nucleic acids of the invention in a pharmaceutically acceptable carrier, as described infra.

In another embodiment, the invention is directed to methods for inhibiting the expression of a TMEFF1 nucleic acid sequence in a prokaryotic or eukaryotic cell comprising providing the cell with an effective amount of a composition comprising an TMEFF1 antisense nucleic acid of the invention.

TMEFF1 antisense nucleic acids and their uses are described in detail below.

The TMEFF1 antisense nucleic acids are of at least six nucleotides and are preferably oligonucleotides (ranging from 6 to about 50 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g. Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/09810, published Dec. 15, 1988) or blood-brain barrier (see, e.g., PCT Publication No. WO 89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549).

In a preferred aspect of the invention, a TMEFF1 antisense oligonucleotide is provided, preferably of single-stranded DNA. In a most preferred aspect, such an oligonucleotide comprises a sequence antisense to the sequence encoding a binding domain of a TMEFF1 protein, most preferably, of a human TMEFF1 protein. The oligonucleotide may be modified at any position on its structure with substituents generally known in the art.

The TMEFF1 antisense oligonucleotide may comprise at least one modified base moiety that is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5 -methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including but not limited to arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, hybridization-triggered cleavage agent, etc.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

In an alternative embodiment, the TMEFF1 antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the TMEFF1 antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the TMEFF1 antisense RNA can be by any promoter known in the art to act in mammalian, preferably human, cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc.

The antisense nucleic acids of the invention comprise a sequence complementary to at least a portion of an RNA transcript of a TMEFF1 gene, preferably a human TMEFF1 gene. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded TMEFF1 antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a TMEFF1 RNA it may contain and still form a stable duplex (or triplex, as the case may be). One skilled in the art can ascertain a tolerable degree of mismatch by use of standard procedures to determine the melting point of the hybridized complex.

In another embodiment, the TMEFF1 antisense oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

5.6.5.2. Ribozymes

In a specific embodiment, the TMEFF1 antisense oligonucleotide comprises catalytic RNA, or a ribozyme. Ribozyme molecules designed to catalytically cleave target gene mRNA transcripts can also be used to prevent translation of target gene mRNA and, therefore, expression of target gene product (see, e.g., PCT International Publication WO 90/11364, published Oct. 4, 1990; Sarver et al., 1990, Science 247:1222-1225).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

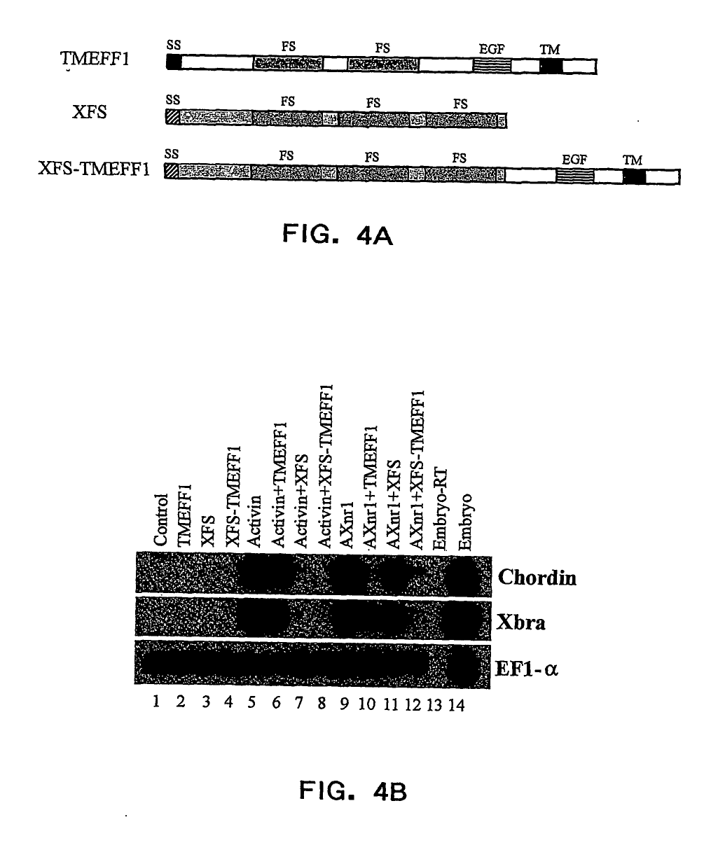

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Cech and collaborators (Zaug, et al., 1984, Science, 224, 574-578; Zaug and Cech, 1986, Science, 231, 470-475; Zaug, et al., 1986, Nature, 324,429-433; published International patent application No. WO 88104300 by University Patents Inc.; Been and Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol m or pol II promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

5.6.5.3. RNA Interference of TMEFF1 Expression

In certain embodiments, an RNA interference (RNAi) molecule is used to decrease TMEFF1 expression. RNA interference (RNAi) is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded (ds) RNA can be used to interfere with gene expression in mammals (Wianny & Zernicka-Goetz, 2000, Nature Cell Biology 2: 70-75; incorporated herein by reference in its entirety). RNAi can be administered as siRNA (small inhibitory RNA)-doublestranded RNAs of 21-25 base pairs, or, alternatively, may be expressed from a vector containing both sense and anti-sense versions of a sequence in vivo such that the dsRNA is formed in vivo. Such expressed dsRNA may be longer than 21-25 base pairs, e.g., 50 to 100 base pairs, 100 to 500 base pairs or 500 to 1000 base pairs in length.

5.6.5.4. Triple Helix Molecules

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC₊ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, in Section 5.8.3, that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

5.6.6. Therapeutic use of TMEFF1 Antisense Nucleic Acids, Ribozymes, RNAI and Triple Helix Molecules

The TMEFF1 antisense nucleic acids, ribozymes or triple helix molecules of the invention can be used to treat (or prevent) disorders of a cell type that expresses, or preferably overexpresses, TMEFF1. In a specific embodiment, such a disorder is a cell proliferation disorder or disorder of a mesodermal or endodermal tissue. In a preferred embodiment, a single-stranded DNA antisense TMEFF1 oligonucleotide is used.

Cell types that express or overexpress TMEFF1 RNA can be identified by various methods known in the art. Such methods include but are not limited to hybridization with a TMEFF1-specific nucleic acid (e.g. by Northern hybridization, dot blot hybridization, in situ hybridization), observing the ability of RNA from the cell type to be translated in vitro into TMEFF1, immunoassay, etc. In a preferred aspect, primary tissue from a patient can be assayed for TMEFF1 expression prior to treatment, e.g., by immunocytochemistry or in situ hybridization.

Pharmaceutical compositions of the invention (see Section 5.8), comprising an effective amount of a TMEFF1 antisense nucleic acid, ribozyme or triple helix molecule in a pharmaceutically acceptable carrier, can be administered to a patient having a disease or disorder that is of a type that expresses or overexpresses TMEFF1 RNA or protein.

The amount of TMEFF1 antisense nucleic acid, ribozyme or triple helix molecule that will be effective in the treatment of a particular disorder or condition will depend on the nature of the disorder or condition, and can be determined by standard clinical techniques. Where possible, it is desirable to determine in vitro the cytotoxicity of the amount of the TMEFF1 antisense nucleic acid, ribozyme or triple helix molecule in a selected cell or tissue to be treated, and then in useful animal model systems prior to testing and use in humans.

In a specific embodiment, pharmaceutical compositions comprising TMEFF1 antisense nucleic acids, ribozymes or triple helix molecules are administered via liposomes, microparticles, or microcapsules. In various embodiments of the invention, it may be useful to use such compositions to achieve sustained release of the TMEFF1 antisense nucleic acids, ribozymes or triple helix molecules. In a specific embodiment, it may be desirable to utilize liposomes targeted via antibodies to specific tissue antigens (e.g., see Leonetti et al., 1990, Proc. Natl. Acad. Sci. U.S.A. 87:2448-2451; Renneisen et al, 1990, J. Biol. Chem. 265:16337-16342).

Additional methods that can be adapted for use to deliver a TMEFF1 antisense nucleic acids, ribozymes or triple helix molecules are described in Section 5.6.6.

5.7. Demonstration of Therapeutic or Prophylactic Utility

Toxicity and efficacy of the prophylactic and/or therapeutic protocols of the instant invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-cancer activity of the therapies used in accordance with the present invention also can be determined by using various experimental animal models for the study of cancer such as the scid mouse model, nude mice with human xenografts, and other animal models, such as hamsters, rabbits, etc. known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties.

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed or angiogenesis assays. A lower level of proliferation or survival of the contacted cells indicates that the therapeutic agent is effective to treat the condition in the patient. Alternatively, instead of culturing cells from a patient, therapeutic agents and methods may be screened using cells of a tumor or malignant cell line or an endothelial cell line. Many assays standard in the art The Therapeutics of the invention are preferably tested in vitro, and then in vivo for the desired therapeutic or prophylactic activity, prior to use in humans. For example, in vitro assays that can be used to determine whether administration of a specific Therapeutic is indicated, include in vitro cell culture assays in which a patient tissue sample is grown in culture, and exposed to or otherwise administered a Therapeutic, and the effect of such Therapeutic upon the tissue sample is observed. In one embodiment, where the patient has a malignancy, a sample of cells from such malignancy is plated out or grown in culture, and the cells are then exposed to a Therapeutic.

Activity of Therapeutics of the invention in treatments involving induction, growth, differentiation or maintenance of a mesodermal, endodermal, epidermal, bone or cartilege tissue derivative may be tested first in vitro in cells that are from mesodermal or endodermal tissues or cells that have potential to differentiate into a particular mesodermal or endodermal cell type. In addition, Therapeutics may be tested in assays in Xenopus embryos for ability to alter cell fate, e.g., to promote or inhibit the formation of ectopic mesodermal or endodermal derived tissues, such as, heart, gut, liver, kidney, pancreas, lung, muscle, blood, etc. These Therapeutics may then be tested in an appropriate animal model for diseases or disorders that can be treated or prevented by modulating the induction, growth, differentiation and/or maintenance of that tissue type.

In other embodiments where the Therapeutic modulates induction, growth, differentiation or maintenance of neural, epidermal, mesodermal, or endodermal tissues, many assays standard in the art can be used to assess such epidermal or neural induction; for example, neural and epidermal induction can be assayed by assaying for modulation of neural or epidermal induction in Xenopus embryos, cell culture, etc, by, for example, morphological inspection of cells, by detecting changes in transcriptional activity or the presence of the gene product of known tissue-specific genes. Other animal models for diseases or disorders associated with neural degeneration, lesions, or epidermal tissue, such as psoriasis, wound healing, etc. are known in the art and can be used to assay Therapeutics of the invention.

In various specific embodiments, in vitro assays can be carried out with representative cells of cell types involved in a patient's disorder, to determine if a Therapeutic has a desired effect upon such cell types.

In other specific embodiments, the in vitro assays described supra can be carried out using a cell line, rather than a cell sample derived from the specific patient to be treated, in which the cell line is derived from or displays characteristic(s) associated with the neural induction disorder desired to be treated or prevented, or is derived from the cell type upon which an effect is desired, according to the present invention.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used.

5.8. Therapeutic/Prophylactic Administration and Compositions

The invention provides methods of treatment (and prophylaxis) by administration to a subject of an effective amount of a Therapeutic of the invention. In a preferred aspect, the Therapeutic is substantially purified. The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the Therapeutic comprises a nucleic acid are described in Section 5.6, above; additional appropriate formulations and routes of administration can be selected from among those described hereinbelow.

Various delivery systems are known and can be used to administer a Therapeutic of the invention, e.g., encapsulation in liposomes, microparticles, microcapsules, recombinant cells capable of expressing the Therapeutic, receptor-mediated endocytosis (see, e.g., Wu and Wu, 1987, J. Biol. Chem. 262:4429-4432), construction of a Therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the Therapeutic can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp.353-365 (1989); Lopez-Berestein, ibid., pp. 317-327; see generally ibid.)

In yet another embodiment, the Therapeutic can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the Therapeutic is a nucleic acid encoding a protein Therapeutic, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide that is known to enter the nucleus (see e.g., Joliot et al., 1991, Proc. Natl. Acad. Sci. USA 88:1864-1868), etc. Alternatively, a nucleic acid Therapeutic can be introduced intracellularly and incorporated within host cell DNA for expression, by homologous recombination.

The present invention also provides pharmaceutical compositions. Such compositions comprise a therapeutically effective amount of a Therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The Therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the Therapeutic of the invention that will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

5.9. Diagnosis and Screening

TMEFF1 proteins, analogues, derivatives, and subsequences thereof, TMEFF1 nucleic acids (and sequences complementary thereto), anti-TMEFF1 antibodies, have uses in diagnostics. Such molecules can be used in assays, such as immunoassays, to detect, prognose, diagnose, or monitor various conditions, diseases, and disorders affecting TMEFF1 expression, in particular that involve TGFβ signalling, particularly nodal, Vg1 and BMP-2 signalling, or monitor the treatment thereof. In particular, such an immunoassay is carried out by a method comprising contacting a sample derived from a patient with an anti-TMEFF1 antibody under conditions such that immunospecific binding can occur, and detecting or measuring the amount of any immunospecific binding by the antibody. In a specific aspect, such binding of antibody, in tissue sections, can be used to detect aberrant TMEFF1 localization or aberrant (e.g., low or absent) levels of TMEFF1. In a specific embodiment, antibody to TMEFF1 can be used to assay in a patient tissue or serum sample for the presence of TMEFF1 where an aberrant level of TMEFF1 is an indication of a diseased condition. By “aberrant levels,” is meant increased or decreased levels relative to that present, or a standard level representing that present, in an analogous sample from a portion of the body or from a subject not having the disorder.

The immunoassays that can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few.

TMEFF1 genes and related nucleic acid sequences and subsequences, including complementary sequences, can also be used in hybridization assays. TMEFF1 nucleic acid sequences, or subsequences thereof comprising about at least 8 nucleotides, can be used as hybridization probes. Hybridization assays can be used to detect, prognose, diagnose, or monitor conditions, disorders, or disease states associated with aberrant changes in TMEFF1 expression and/or activity as described supra. In particular, such a hybridization assay is carried out by a method comprising contacting a sample containing nucleic acid with a nucleic acid probe capable of hybridizing to TMEFF1 DNA or RNA, under conditions such that hybridization can occur, and detecting or measuring any resulting hybridization.

In specific embodiments, diseases and disorders involving defects in TGFβ, particularly, nodal, Vg1 or BMP-2, signalling, e.g., relating to mesodermal, endodermal, neural or epidermal cell induction, growth, differentiation or maintenance can be diagnosed, or their suspected presence can be screened for, or a predisposition to develop such disorders can be detected, by detecting decreased (or increased) levels of TMEFF1 protein, TMEFF1 RNA, or TMEFF1 functional activity (e.g., inhibition of nodal, Vg1 or BMP-2 signalling, etc.), or by detecting mutations in TMEFF1 RNA, DNA or protein (e.g., translocations in TMEFF1 nucleic acids, truncations in the TMEFF1 gene or protein, changes in nucleotide or amino acid sequence relative to wild-type TMEFF1) that cause decreased (or increased) expression or activity of TMEFF1. Such diseases and disorders include but are not limited to those described in Section 5.6. By way of example, levels of TMEFF1 protein can be detected by immunoassay, levels of TMEFF1 RNA can be detected by hybridization assays (e.g., Northern blots, dot blots), TMEFF1 binding to a binding partner can be done by binding assays commonly known in the art, translocations and point mutations in TMEFF1 nucleic acids can be detected by Southern blotting, RFLP analysis, PCR using primers that preferably generate a fragment spanning at least most of the TMEFF1 gene, sequencing of the TMEFF1 genomic DNA or cDNA obtained from the patient, etc.

Kits for diagnostic use are also provided, that comprise in one or more containers an anti-TMEFF1 antibody, and, optionally, a labeled binding partner to the antibody. Alternatively, the anti-TMEFF1 antibody can be labeled (with a detectable marker, e.g., a chemiluminescent, enzymatic, fluorescent, or radioactive moiety). A kit is also provided that comprises in one or more containers a nucleic acid probe capable of hybridizing to TMEFF1 RNA. In a specific embodiment, a kit can comprise in one or more containers a pair of primers (e.g., each in the size range of 6-30 nucleotides) that are capable of priming amplification [e.g., by polymerase chain reaction (see e.g., Innis et al., 1990, PCR Protocols, Academic Press, Inc., San Diego, Calif.), ligase chain reaction (see EP 320,308) use of Qβ replicase, cyclic probe reaction, or other methods known in the art] under appropriate reaction conditions of at least a portion of a TMEFF1 nucleic acid. A kit can optionally further comprise in a container a predetermined amount of a purified TMEFF1 protein or nucleic acid, e.g., for use as a standard or control.

5.10 Screening for TMEFF1 Agonists and Antagonists

TMEFF1 nucleic acids, proteins, and derivatives also have uses in screening assays to detect molecules that specifically bind to TMEFF1 nucleic acids, proteins, or derivatives and thus have potential use as agonists or antagonists of TMEFF1, in particular, molecules that affect cell proliferation, induction, growth, differentiation or maintenance of mesodermal or endodermal tissues, epidermal cell growth, differentiation or maintenance, or neural cell induction, growth, differentiation or maintenance. In a preferred embodiment, such assays are performed to screen for molecules with potential utility as anti-cancer drugs or lead compounds for drug development. The invention thus provides assays to detect molecules that specifically bind to TMEFF1 nucleic acids, proteins, or derivatives. For example, recombinant cells expressing TMEFF1 nucleic acids can be used to recombinantly produce TMEFF1 proteins in these assays, to screen for molecules that bind to a TMEFF1 protein. Molecules (e.g., putative binding partners of TMEFF1) are contacted with the TMEFF1 protein (or fragment thereof) under conditions conducive to binding, and then molecules that specifically bind to the TMEFF1 protein are identified. Similar methods can be used to screen for molecules that bind to TMEFF1 derivatives or nucleic acids. Methods that can be used to carry out the foregoing are commonly known in the art.

By way of example, diversity libraries, such as random or combinatorial peptide or nonpeptide libraries can be screened for molecules that specifically bind to TMEFF1. Many libraries are known in the art that can be used, e.g., chemically synthesized libraries, recombinant (e.g., phage display libraries), and in vitro translation-based libraries.

Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian, R.B., et al., 1992, J. Mol. Biol. 227:711-718); Lenstra, 1992, J. Immunol. Meth. 152:149-157; Kay et al., 1993, Gene 128:59-65; and PCT Publication No. WO 94/18318 dated Aug. 18, 1994.

In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

Screening the libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat No. 5,223,409, and U.S. Pat. No. 5,198,346, all to Ladner et al.; Rebar and Pabo, 1993, Science 263:671-673; and PCT Publication No. WO 94/18318.

In a specific embodiment, screening can be carried out by contacting the library members with a TMEFF1 protein (or nucleic acid or derivative) immobilized on a solid phase and harvesting those library members that bind to the protein (or nucleic acid or derivative). Examples of such screening methods, termed “panning” techniques are described by way of example in Parmley and Smith, 1988, Gene 73:305-318; Fowlkes et al., 1992, BioTechniques 13:422-427; PCT Publication No. WO 94/18318; and in references cited hereinabove.

In another embodiment, the two-hybrid system for selecting interacting proteins in yeast (Fields and Song, 1989, Nature 340:245-246; Chien et al., 1991, Proc. Natl. Acad. Sci. USA 88:9578-9582) can be used to identify molecules that specifically bind to a TMEFF1 protein or derivative.

5.11. Animal Models

The invention also provides animal models for use in screening for molecules that alter TMEFF1 activity.

In one embodiment, animal models for diseases and disorders involving nodal, Vg1 and/or BMP-2 signaling are provided. Such an animal can be initially produced by promoting homologous recombination between a TMEFF1 gene in its chromosome and an exogenous TMEFF1 gene that has been rendered biologically inactive preferably by insertion of a heterologous sequence, e.g., an antibiotic resistance gene). In a preferred aspect, this homologous recombination is carried out by transforming embryo-derived stem (ES) cells with a vector containing the insertionally inactivated TMEFF1 gene, such that homologous recombination occurs, followed by injecting the ES cells into a blastocyst, and implanting the blastocyst into a foster mother, followed by the birth of the chimeric animal (“knockout animal”) in which a TMEFF1 gene has been inactivated (see Capecchi, 1989, Science 244:1288-1292). The chimeric animal can be bred to produce additional knockout animals. Such animals can be mice, hamsters, sheep, pigs, cattle, etc., and are preferably non-human mammals. In a specific embodiment, a knockout mouse is produced.

Such knockout animals are expected to develop or be predisposed to developing diseases or disorders involving neural induction and thus can have use as animal models of such diseases and disorders, e.g., to screen for or test molecules for the ability to inhibit or promote neural induction and thus treat or prevent such diseases or disorders.

In a different embodiment of the invention, transgenic animals that have incorporated and express a functional TMEFF1 gene have use as animal models of diseases and disorders involving deficiencies in neural induction or in which neural induction is desired: Such animals can be used to screen for or test molecules for the ability to promote neural induction and thus treat or prevent such diseases and disorders.

The host cells that are engineered to contain a TMEFF1 gene can also be used to produce nonhuman transgenic animals. For example, in one embodiment, a host cell is a fertilized oocyte or an embryonic stem cell into which a sequence encoding a TMEFF1 polypeptide has been introduced. Such host cells can then be used to create non-human transgenic animals in which exogenous sequences encoding a TMEFF1 polypeptide have been introduced into their genome or homologous recombinant animals in which endogenous genes encoding TMEFF1 have been altered (i.e., knock-outs). Such animals are useful for studying the function and/or activity of the polypeptide and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, such as Xenopus , fish, such as zebra fish and fugu etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a polypeptide of the invention (or a homologue thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a TMEFF1 gene into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

### 6. EXAMPLE

TMEFF1 Inhibits Nodal, VG1 and BMP-2 But Not Activin Signalling

6.1. Introduction

During early vertebrate development, members of the transforming growth factor beta (TGFβ) family play important roles in a variety of processes, including germ layer specification and patterning, cell differentiation and migration, and organogenesis. The activities of TGF-βs need to be tightly controlled to ensure their function at the right time and place. Despite identification of multiple regulators of Bone Morphogenetic Protein (BMP) subfamily ligands, modulators of the activin/nodal class of TGFβ ligands are limited, and include follistatin, Cerberus and Lefty. Recently, a membrane protein, tomoregulin-1 (TMEFF1, originally named X7365), was isolated and found to contain two follistatin modules in addition to an Epidermal Growth Factor (EGF) domain, suggesting that TMEFF1 may participate in regulation of TGFβ function. It is shown herein that unlike follistatin and follistatin-related gene (FLRG), TMEFF1 selectively inhibits nodal but not activin in Xenopus . Interestingly, both the follistatin modules and the EGF domain are required for nodal inhibition. In addition, a soluble protein containing the entire extracellular domain is not sufficient to regulate nodal activity; the location of TMEFF1 at the membrane is essential for its function. These results suggest that TMEFF1 inhibits nodal through a novel mechanism. Consistent with its activity, overexpression of TMEFF1 blocks anterior development of early frog embryos, a phenotype similar to that induced by a dominant negative nodal ligand. Unlike TMEFF1 in mouse, however, where it is expressed widely in pre-gastrula embryos, TMEFF1 in Xenopus is expressed from mid gastrulation onward and is enriched in neural tissue derivatives. The expression pattern indicates that TMEFF1 is not involved in the initial function of nodal during germ layer formation/patterning, but may be required at later stages to modulate nodal activities in neural patterning. In summary, these data demonstrate that tomoregulin-1 is a novel regulator of nodal signaling during early vertebrate embryogenesis.

6.2. Materials and Methods

Construction of TMEFF1 mutants.

All the mutants were constructed with PCR cloning strategy. For TMEFF1-ΔC, PCR with TMEFF-N: GGGAATTCACCATGGATGGATTGCACCCT (SEQ ID NO.:1) and ΔC-C: GGCTCGAGCTAAATACACATGACAATTGC (SEQ ID NO.:2). The PCR fragment was digested with EcoRI and XhoI and inserted into the pCS2++ vector. For TMEFF1-ΔTC, PCR with TMEFF-N and ΔTC-C: GGAGATCTGGTGAGCTTTTGCCTACTTGG (SEQ ID NO.:3); for TMEFF-FS, PCR with TMEFF-N and TRFS-C: GGAGATCTTGTTTCTATACAGCTCCGTAT (SEQ ID NO.:4). Both PCR products were digested with EcoRI and BglII, ligated with BamHI/XbaI fragment of pCS2+MT, and inserted into the EcoRI/XbaI sites of pCS2++ vector. For TMEFF1-ΔFS, PCR with EGF-N: CGCCACAATGGCATAGAAACAGATGAAACA (SEQ ID NO.:5) and TMEFF-C: GGAAGATCTCACCATCCGGGAAGAAGTATC (SEQ ID NO.:6). Cut the PCR product with BglI and BglII, ligate with HindIII/BglI fragment of pCS2++ TMEFF1, inserted into the HindIII/BamHI sites of pCS+MT. For TMEFF1-ΔEGF, PCR with TM-N: GAAGGCCTTTATGTGGTTCCAAGTAGG (SEQ ID NO.:7) and TMEFF-C, double digestion with StuI and BglII, ligate with BamHI/XbaI fragment of pCS2+MT, inserted into the StuI/Xbal sites of pSC2++ vector. For XFS-TMEFF1, PCR on follistatin template with XFS-N: GCGGAATTCACCATGTTAAATGAAAGGATC (SEQ ID NO.:8) and XFS-C: GCGAAGCTTCTTACAGTTGCAAGATCCACT (SEQ ID NO.:9). Digest with EcoRI and HindIII PCR on TMEFF1 template with TMEFF-EGFN: GCGAAGCTTATAGAAACAGATGAAACAAGC (SEQ ID NO.:10) and TMEFF-C. Digest with HindIII and BglII. Ligate the two digested PCR products and the BamHI/XbaI fragment of pCS2+MT, inserted into the EcoRI/XbaI sites of the pCS2++ vector.

RT-PCR.

For TMEFF1 temporal expression, the following two primers are used for RT-PCR assay: TMEFF1-U: TGTGTCTGTAACATTGACTG (SEQ ID NO.:11) and TMEFF1-D: CAGTATTGGCCTGTGTACCC (SEQ ID NO.:12). Primers used for other markers are as described previously (Chang et al., 1997).

Whole mount in situ hybridization.

In situ hybridization was performed as described (Harland, 1991). The TMEFF1 probe was synthesized with T7 polymerase, using NotI linearized pBSKS-TMEFF1 template.

6.3. Results

Selective Inhibition of Nodal But not Activin by TMEFF1

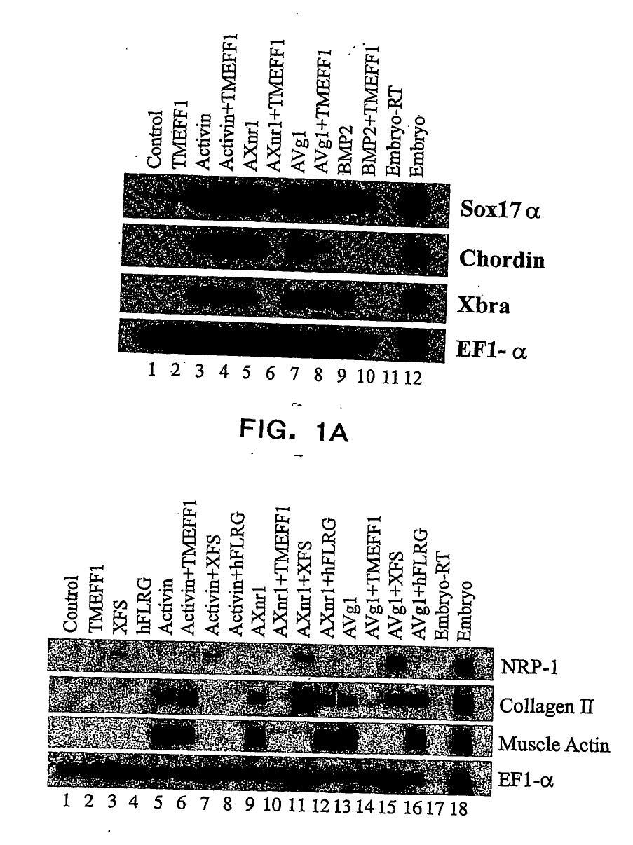

Since follistatin and follistatin-related gene can inhibit activin activities, and TMEFF1 contains two FS modules in its extracellular domain, the ability of TMEFF1 to inhibit activin was investigated. RNAs encoding TMEFF1 and activin were co-injected into the animal poles of two cell stage embryos. The ectodermal explants (animal caps) of injected embryos were dissected at blastula stages and gene expression in these explants was analyzed at gastrula stages by RT-PCR. As shown in FIG. 1A, TMEFF1 does not block activities of activin (lanes 3 and 4). Both mesodermal markers, such as Brachyury (Xbra, Smith et al., 1991) and chordin (Sasai et al., 1994), and endodermal markers, such as Sox17α (Hudson et al., 1997), are induced by activin even in the presence of TMEFF1. To see whether TMEFF1 can influence the activities of other TGFβ ligands, TMEFF1 was co-expressed with the following molecules: AXnr1 (Piccolo et al., 1999) and AVg1 (Kessler and Melton, 1995), chimeric proteins with the activin pro-domain conjugated to Xnr1 and Vg1 mature peptides respectively, and BMP2. Strikingly, TMEFF1 strongly inhibits AXnr1 (lanes 5 and 6), and it also weakly blocks AVg1 and BMP2 (lanes 7 to 10, FIG. 1A). The results suggest that TMEFF1 can regulate TGβ signaling; but unlike follistatin, it selectively inhibits nodal but not activin activities.

Since nodal-related ligands but not activin can be inhibited by the soluble antagonist Cerberus (Piccolo et al., 1999), the possibility that TMEFF1 might block nodal function through induction of Cerberus in animal caps was examined. RT-PCR analysis demonstrates that Cerberus expression is not activated by TMEFF1 in animal caps (data not shown). The data indicate that TMEFF1 does not regulate nodal signaling through the known secreted nodal antagonist.

To further compare the ligand specificity for proteins containing the FS-modules, we coexpressed follistatin (XFS), TMEFF1 and a follistatin-related gene (FLRG) with the TGFβ ligands activin, AXnr1 or AVg1 in early Xenopus embryos. Animal caps of injected embryos were removed at blastula stages and gene expression patterns were analyzed at tailbud stages by RT-PCR. As shown in FIG. 1B, these three FS-motif-containing proteins have different specificities toward the TGFβ ligands. FLRG is a specific antagonist of activin and does not interfere with mesoderm induction by AXnr1 and AVg1 (FIG. 1B, compare lanes 8, 12 and 16 with lanes 5, 9 and 13, respectively). Similarly, follistatin inhibits activin completely; in addition, however, it also dorsalizes mesoderm induction by AXnr1 and AVg1. The axial mesodermal marker, type II collagen, which is expressed in the notochord, is induced by these ligands in the presence of follistatin, while the paraxial muscle marker, muscle actin, is inhibited (FIG. 1B, compare lanes 11 and 15 with lanes 9 and 13, respectively). Follistatin also induces neural tissues, as the expression of the neural marker NRP-1 is stimulated in the caps (FIG. 1B). Dorsalization of mesoderm and neural induction by follistatin likely reflect the ability of follistatin to block BMP activities (Iemura et al.,. 1998). Consistent with the above results in FIG. 1A, TMEFF1 does not inhibit activin, but it does suppress gene activation by AXnr1 and AVg1 (FIG. 1B). Interestingly, though TMEFF1 inhibits mesoderm induction by BMP2 (FIG. 1A), it does not induce neural markers in animal caps (FIG. 1B). TMEFF1, however, does induce markers for cement gland, a structure outside of the anterior neural tissue and whose formation requires partial inhibition of BMPs (Wilson et al., 1997; data not shown). The result suggests that TMEFF1 may not be a strong BMP inhibitor in animal caps. These data thus indicate that the three proteins with follistatin modules have differential function in regulation of TGF, signaling.

Soluble TMEFF1 loses the Nodal Inhibitory Activity

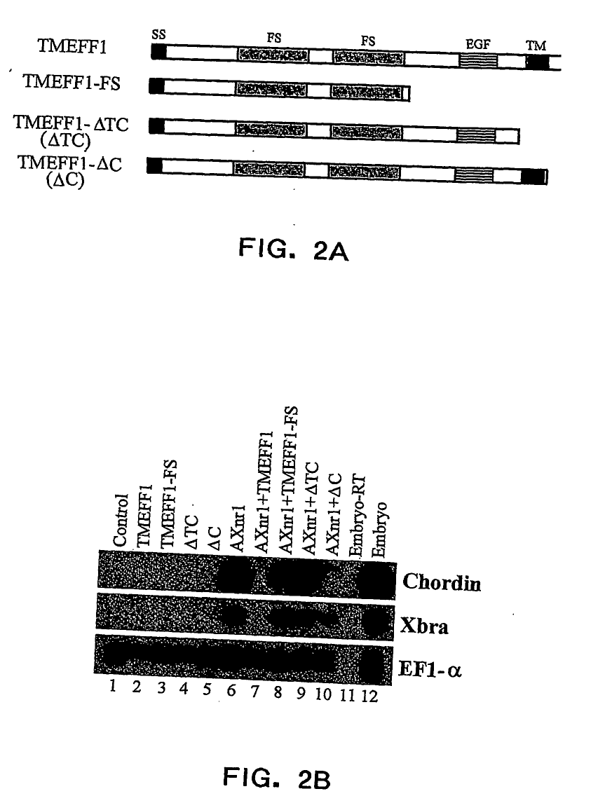

Both follistatin and FLRG inhibit activin through directly binding to the ligand and preventing it from interaction with the activin receptors (Kogawa et al., 1991; Fukui et al., 1993; Tsuchida et al., 2001; Bartholin et al., 2002). The follistatin modules in these proteins may play an important role in ligand binding and the inhibitory activities. Whether the follistatin modules in tomoregulin-1 were sufficient for nodal inhibition was investigated. A mutant TMEFF1 that contains only the two follistatin motifs (TMEFF1-FS, FIG. 2A) was made and coexpressed with AXnr1 in early Xenopus embryos. Assay for gene expression of the ectodermal explants from injected embryos shows that this mutant is not able to block AXnr1 activities in animal caps. All the markers induced by AXnr1 are still expressed in the presence of TMEFF1-FS (compare lane 8 with lane 6, FIG. 2B). The data suggest that additional sequences are required for the inhibitory function of TMEFF1. To see whether the extracellular domain, which contains both the follistatin and the EGF motifs, is sufficient for nodal inhibition, a mutant protein with C-terminal deletion that eliminates the transmembrane region and the C-terminal cytoplasmic tail (TMEFF1-ΔATC, FIG. 2A) was constructed. This mutant, TMEFF1-ΔATC, can be secreted from oocytes injected with its RNA, suggesting that it is a soluble protein (data not shown). Coexpression of TMEFF1-ΔATC with AXnr1 in ectodermal explants shows that like TMEFF1-FS, it does not block gene activation by AXnr1 (lane 9, FIG. 2B). This result demonstrates that secreted TMEFF1 does not inhibit nodal function, and that TMEFF1 may need to be located at the membrane to function as a nodal inhibitor.

TMEFF1 from all species contain a conserved cytoplasmic tail, which may be important for nodal inhibition (Eib and Martens, 1996; Uchida et al., 1999; Eib et al., 2000; Da Silva et al., 2001). To see whether the cytoplasmic region is required for TMEFF1 function, we constructed a mutant TMEFF1 that has deletion in the cytoplasmic domain right after the transmembrane region (TMEFF1-ΔAC, FIG. 2A). Coexpression of TMEFF1-ΔC with AXnr1 shows that this mutant regains the nodal inhibitory activities (lane 10, FIG. 2B). These data thus demonstrate that unlike the soluble proteins follistatin and FLRG, membrane location of TMEFF1 is essential for its function.

Both the FS Modules and the EGF Motif are Required for Nodal Inhibition

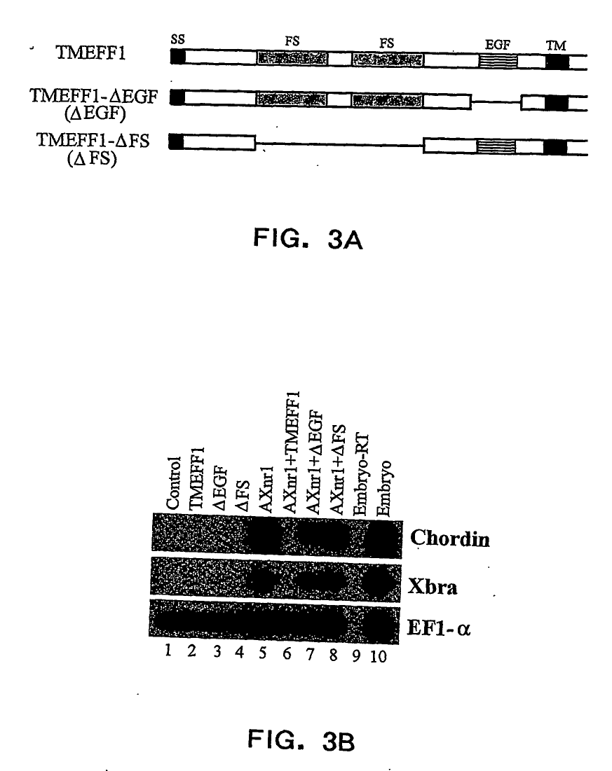

Since soluble TMEFF1 is not sufficient for nodal inhibition and the membrane location is required for TMEFF1 function, it is possible that TMEFF1 interacts with certain membrane proteins that are involved in nodal signal transduction. This interaction may require only the follistatin modules or the EGF motif; or alternatively both domains may be required. To examine whether the follistatin modules and the EGF domain are required for tomoregulin activity, two additional mutant proteins were constructed that have the EGF or the FS domains removed, respectively (TMEFF1-□EGF and TMEFF1-□FS, FIG. 3A). As shown in FIG. 3B, deletion of either the FS modules or the EGF motif impairs the ability of TMEFF1 to block nodal function (compare lanes 7 and 8 with lane 6). The data show that in addition to follistatin modules, the EGF motif also contributes to nodal inhibitory activity of TMEFF1. These data thus suggest that TMEFF1 inhibits nodal through a novel mechanism.

Inhibition of Nodal Activity by Follistatin-TMEFF1 Chimeric Protein

The follistatin modules have been proposed to mediate the TGFα regulatory activities of FS module-containing proteins. The differential regulation of activin and nodal by follistatin and TMEFF1 may therefore depend on the sequence difference in their follistatin motifs. To examine this possibility, a chimeric protein XFS-TMEFF1 was constructed, in which the FS modules of TMEFF1 is replaced with the FS modules of follistatin (FIG. 4A). XFS-TMEFF1 was co-expressed with either activin or AXnr1 in early Xenopus embryos and analyzed gene expression induced by these ligands in the presence of the chimeric protein. Interestingly, as shown in FIG. 4B, XFS-TMEFF1 not only retains the nodal inhibitory activity of TMEFF1, but it also acquires the activin inhibitory function of follistatin. This result suggests that the FS modules in TMEFF1 may not be critical in determination of ligand specificity on nodal. The data again imply that TMEFF1 regulates nodal function through a novel mechanism.

Overexpression of TMEFF1 in Early Xenopus Embryos Induces Anterior Defects

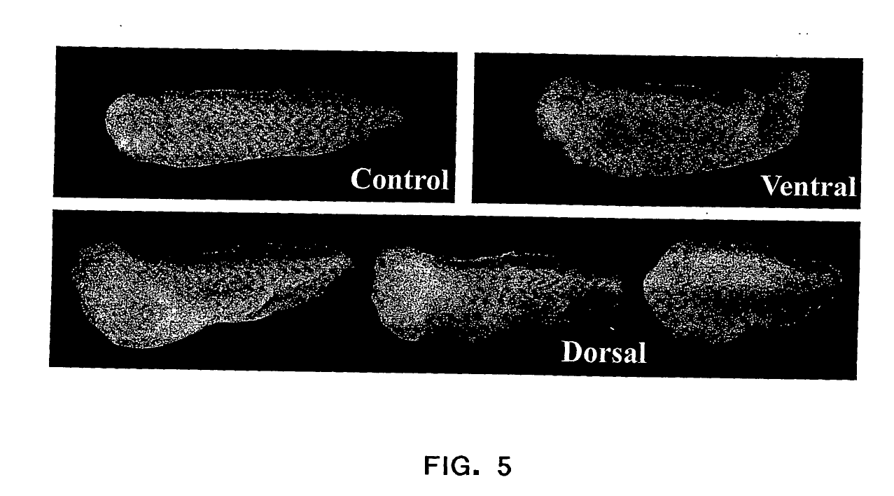

Nodal signaling is required for endoderm and mesoderm formation in early frog embryos; interference with this signaling pathway hinders mesendoderm development and leads to anterior truncation of frog tadpoles (Osada et al., 1999; Agius et al., 2000). To see whether TMEFF1 can also inhibit nodal signaling in whole embryos to induce a phenotype similar to that induced by other nodal antagonists, TMEFF1 RNA was injected into either two dorsal or two ventral blastomeres of 4-cell stage embryos. As shown in FIG. 5, while ventral expression of TMEFF1 causes mild tail truncation, overexpression of TMEFF1 on the dorsal side induces anterior defects in frog embryos, leading to reduction or even absence of head structures. The morphology of the tadpoles is similar to that induced by expression of a dominant-negative nodal ligand (Osada et al., 1999), implying that TMEFF1 can also inhibit nodal in whole frog embryos.

Expression of TMEFF1 During Early Xenopus Development

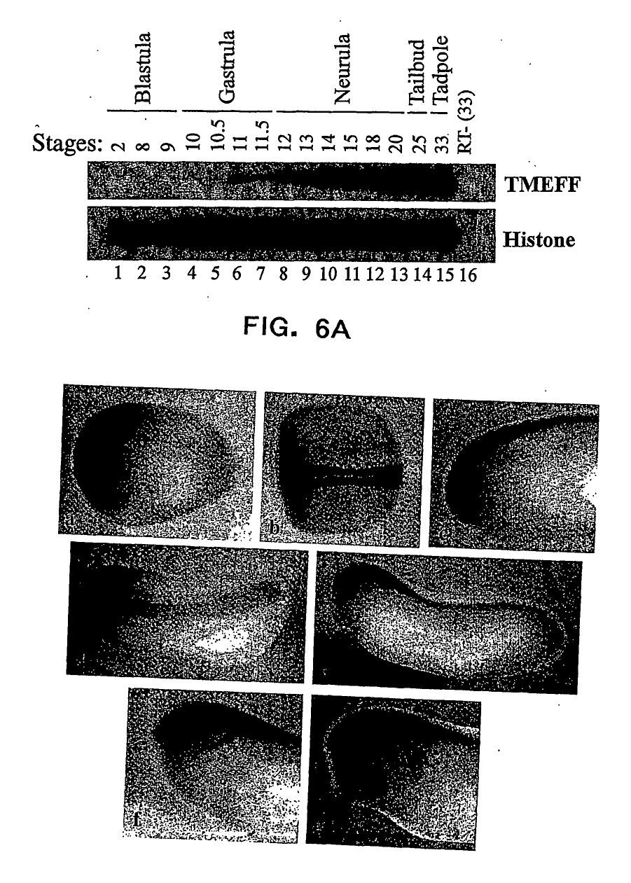

TMEFF1 was originally identified as a gene that was expressed in the hypothalamo-hypophysial axis of adult Xenopus brains (Eib and Martens, 1996). The expression of the gene at early developmental stages in frogs has not been reported. To see when and where TMEFF1 is expressed during early frog embryogenesis, we analyzed its temporal expression profile by reverse transcription PCR (RT-PCR) and its spatial distribution by whole mount in situ hybridization. As shown in FIG. 6A, TMEFF1 is first transcribed at mid-gastrula stages (stage 10.5). The expression level increases during neurula stages and retains to at least tadpole stages (FIG. 6A). During early neurulation, TMEFF1 transcripts are detected in the neural plate (panel a, FIG. 6B). As neurulation proceeds, its messenger RNA is restricted to the neural folds and the dorsal neural tube in the trunk region (panels b-d, FIG. 6B). At tailbud stages, TMEFF1 is expressed in the diencephalon, midbrain, hindbrain (panel f), otic vesicle and the cranial nerve placodes (panels e and g, FIG. 6B) in addition to the trunk dorsal neural tissues. The temporal and spatial expression pattern suggests that TMEFF1 may not participate in early function of nodal in germ layer formation, but may regulate TGFP signaling during neural development.

6.4 Discussion

During early vertebrate development, TGFβ signals regulate many aspects of embryonic induction and patterning. The activities of TGFβs are under the control of both positive and negative factors (Hill, 2001). Follistatin, which is a negative regulator of both activin and BMPs, contains three repetitive modules that may be important for its inhibitory function. Proteins harboring different numbers of follistatin modules have been identified recently. FLRG, which has two FS motifs, acts similarly to follistatin in that it binds to activin with high affinity and blocks activin signaling. The activities of Flik/FRPs, which contain a single FS module, in regulation of TGFP signals, however, have not been documented in detail. Here, we show that TMEFF1, a protein with two FS modules, regulates nodal but not activin activities. This is the first example in which proteins with follistatin motifs can regulate nodal function. These data, however, demonstrate that though required, FS modules in TMEFF1 are not sufficient for nodal inhibition. In fact, not only the second structural motif, the EGF domain, is required, but also the site of expression of the protein at the membrane is necessary for nodal inhibition. These results thus suggest that TMEFF1 regulates nodal activities through a novel mechanism.

In summary, these studies demonstrate that (TMEFF1) can selectively inhibit nodal function. The membrane location of TMEFF1 is important for its inhibitory activity, and both the follistatin modules and the EGF domain are required. The data suggest that tomoregulin-1 may regulate nodal signaling through a novel mechanism.

6.5. References
Agius, E., Oelgeschlager, M., Wessely, O., Kemp, C. and DeRobertis, E. M. (2000) Endodermal nodal-related signals and mesoderm induction in Xenopus . Development 127, 1173-1183.
Bartholin, L., Maguer-Satta, V., Hayette, S., Martel, S., Gadoux, M., Corbo, L., Magaud, J. P. and Rimokh, R. (2002) Transcription activation of FLRG and follistatin by activin A, through Smad proteins, participates in a negative feedback loop to modulate activin A function. Oncogene 21, 2227-2235.
Branford, W. W., Essner, J. J. and Yost, H. J. (2000) Regulation of gut and heart left-right asymmetry by context-dependent interactions between xenopus lefty and BMP4 signaling. Dev. Biol. 223, 291-306.
Casellas, R. and Hemmati-Brivanlou, A. (1998) Xenopus Smad7 inhibits both the activin and BMP pathways and acts as a neural inducer. Dev. Biol. 198, 1-12.
Chang, C., Wilson, P. A., Mathews, L. S. and Hemmati-Brivanlou, A. (1997) A Xenopus type I activin receptor mediates mesodermal but not neural specification during embryogenesis. Development 124, 827-837.
Cheng, A. M. S., Thisse, B., Thisse, C. and Wright, C. V. E. (2000) The lefty-related factor Xatv acts as a feedback inhibitor of nodal signaling in mesoderm induction and L-R axis development in Xenopus . Development 127, 1049-1061.
Da Silva, S. M., Gates, P. B., Eib, D. W., Martens, G. J. M. and Brockes, J. P. (2001) the expression pattern of tomoregulin-1 in urodele limb regeneration and mouse limb development. Mech. Dev. 104, 125-128.
De Groot, Feijen, A., Eib, D., Zwijsen, A., Sugino, H., Martens, G. and van den Eijnden-van Raaij, A. J. M. (2000) Expression patterns of follistatin and two follistatin-related proteins during mouse development. Int. J. Dev. Biol. 44, 327-330.
Derynck, R. and Feng, X. H. (1997) TGF-beta receptor signaling. Biochim Biophys Acta. 1333, F105-150.
Eib, D. W., Holling, T. M., Zwijsen, A., Dewulf, N., de Groot, E., van den Eijnden-van Raaij, A. J. M., Huylebroeck, D. and Martens, G. J. M. (2000) Expression of the follistatin/EGF-containing transmembrane protein M7365 (tomoregulin-1) during mouse development. Mech. Dev. 97, 167-171.
Eib, D. W. and Martens, G. J. M. (1996) A novel transmembrane protein with epidermal growth factor and follistatin domains expressed in the hypothalamo-hypophysial axis of Xenopus laevis. J. Neurochem. 67, 1047-1055.
Gritsman, K., Zhang, J., Cheng, S., Heckscher, E., Talbot, W. S. and Schier, A. F. (1999) The EGF-CFC protein one-eyed pinhead is essential for nodal signaling. Cell 97, 121-132. Fukui, A., Nakamura, T., Sugino, K., Takio, K., Uchiyama, H., Asashima, M. and Sugino, H. (1993) Isolation and characterization of Xenopus follistatin and activins. Dev. Biol. 159, 131-139.
Hansen, C. S., Marion, C. D., Steele, K., George, S. and Smith, W. C. (1997) Direct neural induction and selective inhibition of mesoderm and epidermis inducers by Xnr3. Development 124, 483-492.
Harland, R. M. (1991) In situ hybridization: an improved whole-mount method for Xenopus embryos. Methods Cell Biol. 36, 685-695.
Harland, R. M. and Gerhart, J. (1997) Formation and function of Spemann's organizer. Annu. Rev. Cell. Dev. Biol. 13, 611-667.
Hata, A., Lagna, G., Massague, J. and Hemmati-Brivanlou, A. (1998) Smad6 inhibits BMP/Smad1 signaling by specifically competing with the Smad4 tumor suppressor. Genes & Dev. 12, 186-197.
Hayette, S., Gadoux, M., Martel, S., Bertrand, S., Tigaud, I., Magaud, J. P. and Rimokh, R. (1998) FLRG (follistatin-elated gene), a new target of chromosomal rearrangement in malignant blood disorders. Oncogene 16, 2949-2954.
Hemmati-Brivanlou, A., Kelly, O. G. and Melton, D. A. (1994) Follistatin, an antagonist of activin, is expressed in the Spemann organizer and displays direct neuralizing activity. Cell 77, 283-295.
Hill, C. S. (2001) TGF-β signaling pathways in early Xenopus development. Curr. Opin. Gene. Dev. 11, 533-540.
Hogan, B. L. M. (1996) Bone morphogenetic proteins: multifunctional regulators of vertebrate development. Genes & Dev. 10, 1580-1594.
Horie, M., Mitsumoto, Y., Kyushiki, H., Kanemoto, N., Watanabe, A., Taniguchi, Y., Nishino, N., Okamoto, T., Kondo, M., Mori, T., Noguchi, K., Nakamura, Y., Takahashi, E. and Tanigami, A. (2000) Identification and characterization of TMEFF2, a novel survival factor for hippocampal and mesencephalic neurons. Genomics 67, 146-152.
Hsu, D. R., Economides, A. N., Wang, X., Eimon, P. M. and Harland, R. M. (1998) The Xenopus dorsalizing factor Gremlin identifies a novel family of secreted proteins that antagonize BMP activities. Mol. Cell 1. 673-683.
Hudson, C., Clements, D., Friday, R. V., Stoft, D. and Woodland, H. R. (1997) Xsox17alpha and -beta mediate endoderm formation in Xenopus . Cell 91, 397-405.
lemura, S., Yamamoto, T. S., Takagi, C., Uchiyama, H., Natsume, T., Shimasaki, S., Sugino, H. and Ueno, N. (1998) Direct binding of follistatin to a complex of bone-morphogenetic protein and its receptor inhibits ventral and epidermal cell fates in early Xenopus embryo. Proc. Natl. Acad. Sci. USA 95, 9337-9342.
Kanemoto N, Horie M, Omori K, Nishino N, Kondo M, Noguchi K, Tanigami A. (2001) Expression of TMEFF1 mRNA in the mouse central nervous system: precise examination and comparative studies of TMEFF1 and TMEFF2. Brain Res. Mol. Brain Res. 86, 48-55. Kessler, D. S. and Melton, D. A. (1995) Induction of dorsal mesoderm by soluble, mature Vg1 protein. Development 121, 2155-2164.
Kogawa, K., Nakamura, T., Sugino, K., Takio, K., Titani, K. and Sugino, H. (1991) Activin-binding protein is present in pituitary. Endocrinology 128, 1434-1440.
Mashimo, J., Maniwa, R., Sugino, H. and Nose, K. (1997) Decrease in the expression of a novel TGF □1-inducible and ras-recision gene, TSC-36, in human cancer cells. Cancer Lett. 113,213-219.
Massague, J. (1998) TGF-beta signal transduction. Annu. Rev. Biochem. 67,753-791. Massague, J. and Chen, Y.-G. (2000) Controlling TGF-□ signaling. Genes Dev. 14, 627-644.
Nakao, A., Afrakhte, M., Moren, A., Nakayama, T., Christian, J. L., Heuchel, R., Itoh, S., Kawabata, M., Heldin, N. E., Heldin, C. H. and ten Dijke, P. (1997) Identification of Smad7, a TGFbeta-inducible antagonist of TGF-beta signalling. Nature 389, 631-635. Nakayama, T., Snyder, M. A., Grewal, S. S., Tsuneizumi, K., Tabata, T. and Christian, J. L. (1998) Xenopus Smad8 acts downstream of BMP-4 to modulate its activity during vertebrate embryonic patterning. Development 125, 857-867.
Okabayashi, K., Shoji, H., Onuma, Y., Nakamura, T., Nose, K., Sugino, H. and Asashima, M. (1999) cDNA cloning and distribution of the Xenopus follistatin-related protein. Biochem. Biophys. Res. Comm. 254,42-48.
Onichtchouk, D., Chen, Y. J., Dosch, R., Gawantka, V., Delius, H., Massague, J. and Niebrs, C. (1999) Silencing of TGF-beta signalling by the pseudoreceptor BAMBI. Nature 401, 480-485.
Osada, S.-I. and Wright, C. V. E. (1999) Xenopus nodal-related singaling is essential for mesendodermal patterning during early embryogenesis. Development 126, 3239-3240. Patel, K., Connolly, D. J., Amthor, H., Nose, K. and Cooke, J. (1996) Cloning and early dorsal axial expression of Flik, a chick follistatin-related gene: evidence for involvement in dorsalization/neural induction. Dev. Biol. 178, p327-342.
Piccolo, S., Sasai, Y., Lu, B. and De Robertis, E. M. (1996) Dorsoventral patterning in Xenopus : inhibition of ventral signals by direct binding of chordin to BMP-4. Cell 86, 589-598.
Piccolo, S., Agius, E., Leyns, L., Bhattacharyya, S., Grunz, H., Bouwmeester, T. and De Robertis, E. M. (1999) The head inducer Cerberus is a multifinctional antagonist of Nodal, BMP and Wnt signals. Nature 397, 707-710.
Reissmann, E., Jomvall, H., Blokzijl, A., Andersson, O., Chang, C., Minchiotti, G., Persico, M. G., Ibanez, C. F. and Brivanlou, A. H. (2001) The orphan receptor ALK7 and the activin receptor ALK4 mediate signaling by nodal proteins during vertebrate development. Genes & Dev. 15,2010-2022.
Sakuma, R., Ohnishi Yi, Y., Meno, C., Fujii, H., Juan, H., Takeuchi, J., Ogura, T., Li, E., Miyazono, K. and Hamada, H. (2002) nhibition of Nodal signalling by Lefty mediated through interaction with common receptors and efficient diffusion. Genes Cells 7, 401-412. Sasai, Y., Lu, B., Steinbeisser, H., Geissert, D., Gont, L. K. and De Robertis, E. M. (1994) Xenopus chordin: a novel dorsalizing factor activated by organizer-specific homeobox genes. Cell 79, 779-790.
Sasai, Y., Lu, B., Steinbeisser, H. and De Robertis, E. M. (1995) Regulation of neural induction by the Chd and Bmp-4 antagonistic patterning signals in Xenopus . Nature 376, 333-336.
Schier, A. F. and Shen, M. M. (1999) Nodal signaling in vertebrate development. Nature 403, 385-389.
Schneyer, A., Tortoriello, D., Sidis, Y., Keutmann, H., Matsuzaki, T. and Holmes, W. (2001) Follistatin-related protein (FSRP): a new member of the follistatin gene family. Mol. Cell. Endocrinol. 180, 33-38.
Shen, M. M. and Schier, A. F. (2000) The EGF-CFC gene family in vertebrate development. Trends. Genet. 16, 303-309.
Smith, J. C., Price, B. M., Green, J. B., Weigel, D. and Herrmann, B. G. (1991) Expression of a Xenopus homolog of Brachyury (T) is an immediate-early response to mesoderm induction. Cell 67, 79-87.
Smith, W. C. and Harland, R. M. (1992) Expression cloning of noggin, a new dorsalizing factor localized to the Spemann organizer in Xenopus embryos. Cell 70, 829-840. Tanegashima, K., Yokota, C., Takahashi. S. and Asashima, M. (2000) Expression cloning of Xantivin, a Xenopus lefty/antivin-related gene, involved in the regulation of activin signaling during mesoderm induction. Mech. Dev. 99, 3-14.
Towers, P., Patel, K., Withington, S., Isaac, A. and Cooke, J. (1999) Flik, a chick follistatin- related gene, functions in gastrular dorsalisation/neural induction and in subsequent maintenance of midline Sonic hedgehog signalling. Dev. Biol. 214, 298-317. Tsuchida, K., Matsuzaki, T., Yamakawa, N., Liu, Z. and Sugino, H. (2001) Intracellular and extracellular control of activin function by novel regulatory molecules. Mol. Cell. Endocrinol. 180, 25-31.
Uchida, T., Wada, K., Akamatsu, T., Yonezawa, M., Noguchi, H., Mizoguchi, A., Kasuga, M. and Sakamoto, C. (1999) A novel Epidermal Growth Factor-like molecule containing two follistatin modules stimulates tyrosine phosphorylation of erbB-4 in MKN28 gastric cancer cells. Biochem. Biophys. Res. Comm. 266, 593-602.
Whitman, M. (2001) Nodal signaling in early vertebrate embryos: themes and variations. Dev. Cell 1, 605-617.
Wilson, P. A., Lagna, G., Suzuki, A. and Hemmati-Brivanlou, A. (1997) Concentration-dependent patterning of the Xenopus ectoderm by BMP4 and its signal transducer Smad1. Development 124, 3177-3184.
Yeo, C.-Y. and Whitman, M. (2001) Nodal signals to Smads through Cripto-dependent and Cripto-independent mechanisms. Mol. Cell 7, 949-957. Zimmerman, L. B., de Jesus-Escobar, J. M. and Harland, R. M. (1996) The Spemann organizer signal noggin binds and inactivates bone morphogenetic protein 4. Cell 86, 599-606.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A method of treating or preventing a disease or disorder involving cell hyperproliferation in a subject, said method comprising administering to a subject in which such treatment or prevention is desired a therapeutically effective amount of a molecule that inhibits TMEFF1 function.

2. The method according to claim 1 in which the disease or disorder is a malignancy.

3. The method according to claim 1 in which the disease or disorder is selected from the group consisting of bladder cancer, breast cancer, colon cancer, leukemia, lung cancer, melanoma, pancreatic cancer, sarcoma, and uterine cancer.

4. The method according to claim 1 in which the subject is a human.

5. The method according to claim 1 in which the disease or disorder is selected from the group consisting of premalignant conditions, benign tumors, hyperproliferative disorders, and benign dysproliferative disorders.

6. The method according to claim 1 in which the molecule that inhibits TMEFF1 function is an anti-TMEFF1 antibody, a TMEFF1 antisense nucleic acid, or TMEFF1 dsRNA.

7. The method according to claim 6 in which the molecule that inhibits TMEFF1 function is an oligonucleotide which (a) consists of at least six nucleotides; (b) comprises a sequence complementary to at least a portion of an RNA transcript of a TMEFF1 gene; and (c) is hybridizable to the RNA transcript under moderately stringent conditions.

8. A method of treating or preventing a disease or disorder in which cell proliferation is desired, said method comprising administering to a subject in which such treatment is desired a therapeutically effective amount of a molecule that promotes TMEFF1 function.

9. The method according to claim 8 in which the molecule that promotes TMEFF1 function is a TMEFF1 protein; a fragment of a TMEFF1 protein containing two FS domains and an EGF domain; a nucleic acid encoding a TMEFF1 protein; and a nucleic acid encoding a fragment of a TMEFF1 protein containing two FS domains and an EGF domain.

10. A method of diagnosing a disease or disorder characterized by an aberrant level of nodal, VG1 or BMP-2 signalling in a subject, said method comprising measuring the level of TMEFF1 RNA or TMEFF1 protein in a sample derived from the subject, in which an increase or decrease in the level of TMEFF1 RNA or protein, relative to the level of TMEFF1 RNA or protein found in an analogous sample from a subject not having the disease or disorder indicates the presence of the disease or disorder in the subject.

11. A method of diagnosing or screening for the presence of or a predisposition for developing a disease or disorder involving aberrant nodal, Vg1 of BMP-2 signalling in a subject, said method comprising measuring the level of TMEFF1 protein, TMEFF1 RNA or TMEFF1 functional activity in a sample derived from the subject, in which a decrease in the level of TMEFF1 protein, TMEFF1 RNA, or TMEFF1 functional activity in the sample, relative to the level of TMEFF1 protein, TMEFF1 RNA, or TMEFF1 functional activity found in an analogous sample from a subject not having the disease or disorder or a predisposition for developing the disease or disorder, indicates the presence of the disease or disorder or a predisposition for developing the disease or disorder.

12. A method of increasing cell growth in animals or plants comprising inhibiting TMEFF1 expression or activity in said animals or plants.

13. The method according to claim 12 in which the molecule that inhibits TMEFF1 function is selected from the group consisting of an anti-TMEFF1 antibody or a TMEFF1 binding fragment or derivative thereof, a TMEFF1 derivative or analog that is capable of being bound by an anti-TMEFF1 antibody, a TMEFF1 antisense nucleic acid, and a nucleic acid comprising at least a portion of a TMEFF1 gene into which a heterologous nucleotide sequence has been inserted such that said heterologous sequence inactivates the biological activity of the at least a portion of the TMEFF1 gene, in which the TMEFF1 gene portion flanks the heterologous sequence so as to promote homologous recombination with a genomic TMEFF1 gene.

14. A method for differentiating mammalian stem cells into endodermal or mesodermal cell types, said method comprising contacting a mammalian stem cell in vitro with a molecule that inhibits TMEFF1 function at a concentration and for a period of time sufficient to cause differentiation of said mammalian stem cell.

15. The method of claim 14, wherein said mammalian cells are embryonic stem cells.

16. The method of claim 15, wherein said embryonic stem cells are human embryonic stem cells.

17. The method of claim 14, wherein said molecule that inhibits TMEFF1 function is an anti-TMEFF1 antibody, a TMEFF-1 antisense nucleic acid, or a TMEFF1 dsRNA.
